(19) United States
(12) Patent Application Publication
(10) Pub. No.: US 2025/0163445 A1
(43) Pub. Date: May 22, 2025

(54) DOMESTICATION OF A LEGUME PLANT

(71) Applicant: BETTERSEEDS LTD, Givat Chen (IL)

(72) Inventors: Ido MARGALIT, Gan Yavne (IL); Tal SHERMAN, Rehovot (IL); Shira COREM, Sdei Hemed (IL)

(21) Appl. No.: 19/025,222

(22) Filed: Jan. 16, 2025

Related U.S. Application Data
(62) Division of application No. 18/279,761, filed on Sep. 1, 2023.
(60) Provisional application No. 63/155,340, filed on Mar. 2, 2021.
(60) Provisional application No. 63/252,182, filed on Oct. 5, 2021.

(51) Int. Cl. C12N 15/82; A01H 6/54; C12N 9/22

(52) U.S. Cl. CPC C12N 15/8262; A01H 6/54; C12N 9/22; C12N 15/8205; C12N 15/8213; C12N 2310/20

(57) ABSTRACT

The present disclosure relates to conferring desirable agronomic traits in Legume plants. More particularly, the current invention pertains to producing Legume plants with improved traits by manipulating genes controlling plant architecture.

### FIELD OF THE INVENTION

The present disclosure relates to conferring desirable agronomic traits in Legumes plants. More particularly, the current invention pertains to producing Legumes plants with improved traits by manipulating genes controlling plant architecture.

### BACKGROUND OF THE INVENTION

One of the most important determinants of crop productivity is plant architecture. For many crops, artificial selection for modified shoot architectures provided critical steps towards improving yield, followed by improvements enabling large-scale field production. A prominent example is tomato, in which the discovery of a mutation in the antiflorigen-encoding self-pruning gene (sp), led to determinate plants that provided a burst of flowering and synchronized fruit ripening, permitting mechanical harvesting.

The publication of Li et al (2018), nature biotechnology, “Domestication of wild tomato is accelerated by genome editing”, teach the assembly of a set of six gRNAs to edit four genes (SlCLV3, SlWUS, SP and SP5G), into one construct. The construct was transformed into four S. pimpinellifolium accessions, all of which are resistant to bacterial spot disease, and two of which are salt tolerant. Small indels and large insertions have been identified in the targeted regulatory regions of SlCLV3 and SlWUS in T0 and their T1 mutant plants. It was reported in this publication that although SP and SP5G are crucial for improving the harvest index, the limited allelic variation has hampered efforts to optimize this trait. It was further reported that locule number was not increased in T0 and T1 plants with large insertions and inversions in the targeted SlCLV3 promoter region. One explanation for this finding is that the targeted region of the S1CLV3 promoter may not be essential for regulating SlCLV3 transcription. Alternatively, it was suggested that disruption of regions (gRNA-5) flanking the CArG element downstream of SlWUS may have decreased its transcription and counteracted the effects of mutation of SlCLV3, owing to a negative feedback loop of CLV3-WUS in controlling stem cell proliferation.

The publication of Zsögön et al (2018), nature biotechnology, “De novo domestication of wild tomato using genome editing”, discloses a devised CRISPR-Cas9 genome engineering strategy to combine agronomically desirable traits with useful traits presented in Solanum pimpinellifolium wild lines. The four edited genes were SELF-PRUNING (SP), OVATE (O), FRUIT WEIGHT 2.2 (FW2.2) and LYCOPENE BETA CYCLASE (CycB).

Lemmon et al (2018), Nature Plants, “Rapid improvement of domestication traits in an orphan crop by genome editing”, describes the usage of CRISPR-Cas9 to mutate orthologues of tomato domestication and improvement genes that control plant architecture, flower production and fruit size in the orphan Solanaceae crop ‘groundcherry’ (Physalis pruinosa).

In open field crops, such as Legumes and Cocoa, it is essential for enabling sustainable agriculture, to be able to harvest the plants mechanically, instead of manual labor. Furthermore, resistance to plant diseases, such as fungi and viruses are also essential in order to maintain profitability and sustainability for open field crops.

Certain open field crops, including soybean, have been domesticated through conventional breeding to express determinate characteristics that enable easier harvesting via machinery.

Certain legumes, such as peanuts and/or cowpea, due to lack of genetic diversity or investment in breeding, do not exhibit this trait.

In view of the above, there is still a long felt and unmet need to manipulate Legume plant architecture in a rapid and efficient way to increase yield and reduce production costs.

### SUMMARY OF THE INVENTION

It is one object of the present invention to disclose a modified Legume plant exhibiting at least one improved domestication trait as compared to a corresponding control Legume plant, wherein said modified plant comprises a mutated SELF PRUNING (SP) Peanut (Arachis hypogaea) gene.

It is another object of the present invention to disclose the modified Legume plant as defined in any of the above, wherein said Peanut (Arachis hypogaea) SP gene is selected from AhSP1-AhSP9 comprising a nucleic acid sequence with at least 75% sequence identity to a sequence selected from SEQ ID NO:1025, SEQ ID NO:1098, SEQ ID NO:1180, SEQ ID NO:1290, SEQ ID NO:1409, SEQ ID NO:1529, SEQ ID NO:1640, SEQ ID NO:1749 and SEQ ID NO:1928, respectively, or a functional variant thereof and any combination thereof.

It is another object of the present invention to disclose the modified Legume plant as defined in any of the above, wherein said mutation is introduced using mutagenesis, small interfering RNA (siRNA), microRNA (miRNA), artificial miRNA (amiRNA), DNA introgression, endonucleases or any combination thereof.

It is another object of the present invention to disclose the modified Legume plant as defined in any of the above, wherein said mutation is introduced using targeted genome modification.

It is another object of the present invention to disclose the modified Legume plant as defined in any of the above, wherein said mutation is introduced using CRISPR (Clustered Regularly Interspaced Short Palindromic Repeats) and CRISPR-associated (Cas) gene (CRISPR/Cas), Transcription activator-like effector nuclease (TALEN), Zinc Finger Nuclease (ZFN), meganuclease or any combination thereof.

It is another object of the present invention to disclose the modified Legume plant as defined in any of the above, wherein said Cas gene is selected from the group consisting of Cas9, Cas12, Cas13, Cas14, CasX, CasY, Csn1, Cpf1 and any combination thereof.

It is another object of the present invention to disclose the modified Legume plant as defined in any of the above, wherein the mutated SP gene is a CRISPR/Cas9-induced heritable mutated allele.

It is another object of the present invention to disclose the modified Legume plant as defined in any of the above, wherein said mutation is a missense mutation, nonsense mutation, insertion, deletion, indel, substitution or duplication.

It is another object of the present invention to disclose the modified Legume plant as defined in any of the above, wherein the insertion or the deletion produces a gene comprising a frameshift.

It is another object of the present invention to disclose the modified Legume plant as defined in any of the above, wherein said plant is homozygous for said at least one mutated SP gene.

It is another object of the present invention to disclose the modified Legume plant as defined in any of the above, wherein said mutation is in the coding region of said gene, a mutation in the regulatory region of said gene, or an epigenetic factor.

It is another object of the present invention to disclose the modified Legume plant as defined in any of the above, wherein said mutation is a silencing mutation, a knockdown mutation, a knockout mutation, a loss of function mutation or any combination thereof.

It is another object of the present invention to disclose the modified Legume plant as defined in any of the above, wherein said mutation is generated in planta.

It is another object of the present invention to disclose the modified Legume plant as defined in any of the above, wherein said mutation is generated in planta via introduction of a construct comprising (a) Cas DNA and gRNA sequence selected from the group consisting of SEQ ID NO:1027-1097, SEQ ID NO:1100-1179, SEQ ID NO:1182-1289, SEQ ID NO:1292-1408, SEQ ID NO:1411-1528, SEQ ID NO:1531-1639, SEQ ID NO:1642-1748, SEQ ID NO:1751-1927 and SEQ ID NO:1930-2051, for said at least one Peanut SP gene, and any combination thereof, or (b) a ribonucleoprotein (RNP) complex comprising Cas protein and gRNA sequence selected from the group consisting of SEQ ID NO:1027-1097, SEQ ID NO:1100-1179, SEQ ID NO:1182-1289, SEQ ID NO:1292-1408, SEQ ID NO:1411-1528, SEQ ID NO:1531-1639, SEQ ID NO:1642-1748, SEQ ID NO:1751-1927 and SEQ ID NO:1930-2051 for said at least one Peanut SP gene, and any combination thereof.

It is another object of the present invention to disclose the modified Legume plant as defined in any of the above, wherein said mutation in said at least one Peanut SP gene is generated in planta via introduction of a construct comprising (a) Cas DNA and gRNA sequence selected from the group consisting of SEQ ID NO:1027-1097, SEQ ID NO:1100-1179, SEQ ID NO:1182-1289, SEQ ID NO:1292-1408, SEQ ID NO:1411-1528, SEQ ID NO:1531-1639, SEQ ID NO:1642-1748, SEQ ID NO:1751-1927 and SEQ ID NO:1930-2051 for Peanut SEQ ID NO:1025, SEQ ID NO:1098, SEQ ID NO:1180, SEQ ID NO:1290, SEQ ID NO:1409, SEQ ID NO:1529, SEQ ID NO:1640, SEQ ID NO:1749 and SEQ ID NO:1928, respectively, and any combination thereof, or (b) a ribonucleoprotein (RNP) complex comprising Cas protein and gRNA sequence selected from the group consisting of SEQ ID NO:1027-1097, SEQ ID NO:1100-1179, SEQ ID NO:1182-1289, SEQ ID NO:1292-1408, SEQ ID NO:1411-1528, SEQ ID NO:1531-1639, SEQ ID NO:1642-1748, SEQ ID NO:1751-1927 and SEQ ID NO:1930-2051 for Peanut SEQ ID NO:1025, SEQ ID NO:1098, SEQ ID NO:1180, SEQ ID NO:1290, SEQ ID NO:1409, SEQ ID NO:1529, SEQ ID NO:1640, SEQ ID NO:1749 and SEQ ID NO:1928, respectively, and any combination thereof.

It is another object of the present invention to disclose the modified Legume plant as defined in any of the above, wherein said gRNA sequence comprises a 3′ NGG Protospacer Adjacent Motif (PAM).

It is another object of the present invention to disclose the modified Legume plant as defined in any of the above, wherein said construct is introduced into the plant cells via Agrobacterium infiltration, virus-based plasmids for delivery of genome editing molecules, or mechanical insertion such as polyethylene glycol (PEG) mediated DNA transformation, electroporation or gene gun biolistics.

It is another object of the present invention to disclose the modified Legume plant as defined in any of the above, wherein said mutation confers reduced expression of said at least one SP gene.

It is another object of the present invention to disclose the modified Legume plant as defined in any of the above, wherein said modified plant has decreased expression levels of said SP gene.

It is another object of the present invention to disclose the modified Legume plant as defined in any of the above, wherein the sequence of said expressed SP gene is selected from the group consisting of: at least 75% identity to any one of Peanut polypeptide SEQ ID NO:1026, SEQ ID NO:1099, SEQ ID NO:1181, SEQ ID NO:1291, SEQ ID NO:1410, SEQ ID NO:1530, SEQ ID NO:1641, SEQ ID NO:1750 and SEQ ID NO:1929, or a functional variant thereof.

It is another object of the present invention to disclose the modified Legume plant as defined in any of the above, wherein said SP gene encodes a polypeptide sequence selected from the group consisting of: at least 75% identity to any one of Peanut polypeptide SEQ ID NO:1026, SEQ ID NO:1099, SEQ ID NO:1181, SEQ ID NO:1291, SEQ ID NO:1410, SEQ ID NO:1530, SEQ ID NO:1641, SEQ ID NO:1750 and SEQ ID NO:1929, or a functional variant thereof.

It is another object of the present invention to disclose the modified Legume plant as defined in any of the above, wherein said modified plant is semi-determinant.

It is another object of the present invention to disclose the modified Legume plant as defined in any of the above, wherein said modified plant has determinant growth habit.

It is another object of the present invention to disclose the modified Legume plant as defined in any of the above, wherein said modified plant flowers earlier than a corresponding control Legume plant lacking said mutated SP gene.

It is another object of the present invention to disclose the modified Legume plant as defined in any of the above, wherein said modified plant exhibits improved earliness as compared to a corresponding control Legume plant lacking said mutated SP gene.

It is another object of the present invention to disclose the modified Legume plant as defined in any of the above, wherein said modified plant exhibits suppressed and/or similar sympodial shoot termination as compared to a corresponding control Legume plant lacking said mutated SP gene.

It is another object of the present invention to disclose the modified Legume plant as defined in any of the above, wherein said domestication trait is selected from the group consisting of reduced flowering time, earliness, synchronous flowering, reduced day-length sensitivity, determinant or semi-determinant architecture, early termination of sympodial cycling, earlier axillary shoot flowering, compact growth habit, reduced height, reduced number of sympodial units, adaptation to mechanical harvest, higher harvest index and any combination thereof.

It is another object of the present invention to disclose a modified Legume plant, plant part, plant tissue or plant cell as defined in any of the above, wherein said plant does not comprise a transgene.

It is another object of the present invention to disclose a plant part, plant cell, plant pod or plant seed of a modified Legume plant as defined in any of the above.

It is another object of the present invention to disclose a tissue culture of regenerable cells, protoplasts or callus obtained from the modified Legume plant as defined in any of the above.

It is another object of the present invention to disclose a method for producing a modified Legume plant exhibiting at least one improved domestication trait compared with a corresponding control Legume, said method comprises steps of genetically modifying at least one Legume SELF PRUNING (SP) gene selected from Peanut (Arachis hypogaea) SP gene, the resultant mutated SP gene has reduced expression level.

It is another object of the present invention to disclose the method as defined in any of the above, wherein said method comprises steps of genetically modifying the at least one Legume SP gene using targeted genome editing introducing a loss of function mutation in the at least one Peanut (Arachis hypogaea) SP gene.

It is another object of the present invention to disclose the method as defined in any of the above, wherein said Peanut (Arachis hypogaea) SP gene is selected from AhSP1-AhSP9 comprising a nucleic acid sequence with at least 75% sequence identity to a sequence selected from SEQ ID NO:1025, SEQ ID NO:1098, SEQ ID NO:1180, SEQ ID NO:1290, SEQ ID NO:1409, SEQ

ID NO:1529, SEQ ID NO:1640, SEQ ID NO:1749 and SEQ ID NO:1928, or a functional variant thereof and any combination thereof.

It is another object of the present invention to disclose the method as defined in any of the above, wherein said method comprises steps of:
a. identifying at least one Legume SP gene in a predetermined Legume plant;
b. synthetizing at least one guide RNA (gRNA) comprising a nucleotide sequence complementary to said at least one identified Legume SP gene;
c. transforming the predetermined Legume plant cells with a construct comprising (a) Cas nucleotide sequence operably linked to said at least one gRNA, or (b) a ribonucleoprotein (RNP) complex comprising Cas protein and said at least one gRNA;
d. screening the genome of said transformed predetermined Legume plant cells for induced targeted loss of function mutation in said at least one Legume SP gene;
e. regenerating Legume plants carrying said loss of function mutation in at least one of said Legume SP gene; and
f. screening said regenerated plants for a Legume plant with improved domestication trait.

It is another object of the present invention to disclose the method as defined in any of the above, wherein said step of screening the genome of said transformed plant cells for induced targeted loss of function mutation further comprises steps of obtaining a nucleic acid sample of said transformed plant and performing a nucleic acid amplification and optionally restriction enzyme digestion to detect a mutation in said at least one of said Legume SP gene.

It is another object of the present invention to disclose the method as defined in any of the above, wherein said SP gene is selected from the group consisting of Peanut SP gene comprising a sequence having at least 75% identity to a sequence selected from SEQ ID NO:1025, SEQ ID NO:1098, SEQ ID NO:1180, SEQ ID NO:1290, SEQ ID NO:1409, SEQ ID NO:1529, SEQ ID NO:1640, SEQ ID NO:1749 and SEQ ID NO:1928, or a functional variant thereof and any combination thereof.

It is another object of the present invention to disclose the method as defined in any of the above, wherein said mutation is introduced using mutagenesis, small interfering RNA (siRNA), microRNA (miRNA), artificial miRNA (amiRNA), DNA introgression, endonucleases or any combination thereof.

It is another object of the present invention to disclose the method as defined in any of the above, wherein said mutation is introduced using targeted genome modification.

It is another object of the present invention to disclose the method as defined in any of the above, wherein said mutation is introduced using CRISPR (Clustered Regularly

Interspaced Short Palindromic Repeats) and CRISPR-associated (Cas) gene (CRISPR/Cas), Transcription activator-like effector nuclease (TALEN), Zinc Finger Nuclease (ZFN), meganuclease or any combination thereof.

It is another object of the present invention to disclose the method as defined in any of the above, wherein said Cas gene is selected from the group consisting of Cas9, Cas12, Cas13, Cas14, CasX, CasY, Csn1, Cpf1 and any combination thereof.

It is another object of the present invention to disclose the method as defined in any of the above, wherein the mutated SP gene is a CRISPR/Cas9-induced heritable mutated allele.

It is another object of the present invention to disclose the method as defined in any of the above, wherein said mutation is a missense mutation, nonsense mutation, insertion, deletion, indel, substitution or duplication.

It is another object of the present invention to disclose the method as defined in any of the above, wherein the insertion or the deletion produces a gene comprising a frameshift.

It is another object of the present invention to disclose the method as defined in any of the above, wherein said plant is homozygous for said at least one Legume SP gene.

It is another object of the present invention to disclose the method as defined in any of the above, wherein said mutation is in the coding region of said gene, a mutation in the regulatory region of said gene, or an epigenetic factor.

It is another object of the present invention to disclose the method as defined in any of the above, wherein said mutation is a silencing mutation, a knockdown mutation, a knockout mutation, a loss of function mutation or any combination thereof.

It is another object of the present invention to disclose the method as defined in any of the above, wherein said mutation is generated in planta.

It is another object of the present invention to disclose the method as defined in any of the above, wherein said mutation is generated in planta via introduction of a construct comprising (a) Cas DNA and gRNA sequence selected from the group consisting of SEQ ID NO:1027-1097, SEQ ID NO:1100-1179, SEQ ID NO:1182-1289, SEQ ID NO:1292-1408, SEQ ID NO:1411-1528, SEQ ID NO:1531-1639, SEQ ID NO:1642-1748, SEQ ID NO:1751-1927 and SEQ ID NO:1930-2051, for said at least one Peanut SP gene, and any combination thereof, or (b) a ribonucleoprotein (RNP) complex comprising Cas protein and gRNA sequence selected from the group consisting of SEQ ID NO:1027-1097, SEQ ID NO:1100-1179, SEQ ID NO:1182-1289, SEQ ID NO:1292-1408, SEQ ID NO:1411-1528, SEQ ID NO:1531-1639, SEQ ID NO:1642-1748, SEQ ID NO:1751-1927 and SEQ ID NO:1930-2051, for said at least one Peanut SP gene, and any combination thereof.

It is another object of the present invention to disclose the method as defined in any of the above, wherein said mutation in said Peanut SP genes is generated in planta via introduction of a construct comprising (a) Cas DNA and gRNA sequence selected from the group consisting of SEQ ID NO:1027-1097, SEQ ID NO:1100-1179, SEQ ID NO:1182-1289, SEQ ID NO:1292-1408, SEQ ID NO:1411-1528, SEQ ID NO:1531-1639, SEQ ID NO:1642-1748, SEQ ID NO:1751-1927 and SEQ ID NO:1930-2051 for Peanut SEQ ID NO:1025, SEQ ID NO:1098, SEQ ID NO:1180, SEQ ID NO:1290, SEQ ID NO:1409, SEQ ID NO:1529, SEQ ID NO:1640, SEQ ID NO:1749 and SEQ ID NO:1928, respectively, or (b) a ribonucleoprotein (RNP) complex comprising Cas protein and gRNA sequence selected from the group consisting of SEQ ID NO:1027-1097, SEQ ID NO:1100-1179, SEQ ID NO:1182-1289, SEQ ID NO:1292-1408, SEQ ID NO:1411-1528, SEQ ID NO:1531-1639, SEQ ID NO:1642-1748, SEQ ID NO:1751-1927 and SEQ ID NO:1930-2051 for Peanut SEQ ID NO:1025, SEQ ID NO:1098, SEQ ID NO:1180, SEQ ID NO:1290, SEQ ID NO:1409, SEQ ID NO:1529, SEQ ID NO:1640, SEQ ID NO:1749 and SEQ ID NO:1928, respectively.

It is another object of the present invention to disclose the method as defined in any of the above, wherein said gRNA sequence comprises a 3′ NGG Protospacer Adjacent Motif (PAM).

It is another object of the present invention to disclose the method as defined in any of the above, wherein said construct is introduced into the plant p cells via Agrobacterium infiltration, virus based plasmids for delivery of the genome editing molecules or mechanical insertion such as polyethylene glycol (PEG) mediated DNA transformation, electroporation or gene gun biolistics.

It is another object of the present invention to disclose the method as defined in any of the above, wherein said modified plant has decreased expression levels of at least one of said Legume SELF PRUNING (SP) gene.

It is another object of the present invention to disclose the method as defined in any of the above, wherein said mutation confers reduced expression of said at least one SP gene.

It is another object of the present invention to disclose the method as defined in any of the above, wherein the sequence of said expressed Legume SELF PRUNING (SP) gene is selected from the group consisting of: at least 75% identity to any one of Peanut polypeptide SEQ ID NO:1026, SEQ ID NO:1099, SEQ ID NO:1181, SEQ ID NO:1291, SEQ ID NO:1410, SEQ ID NO:1530, SEQ ID NO:1641, SEQ ID NO:1750 and SEQ ID NO:1929, or a functional variant thereof.

It is another object of the present invention to disclose the method as defined in any of the above, wherein the Legume SELF PRUNING (SP) gene encodes a polypeptide sequence selected from the group consisting of: at least 75% identity to any one of Peanut polypeptide SEQ ID NO:1026, SEQ ID NO:1099, SEQ ID NO:1181, SEQ ID NO:1291, SEQ ID NO:1410, SEQ ID NO:1530, SEQ ID NO:1641, SEQ ID NO:1750 and SEQ ID NO:1929, or a functional variant thereof.

It is another object of the present invention to disclose the method as defined in any of the above, wherein said modified plant is semi-determinant.

It is another object of the present invention to disclose the method as defined in any of the above, wherein said modified plant has determinant growth habit.

It is another object of the present invention to disclose the method as defined in any of the above, wherein said modified plant flowers earlier than a corresponding control Legume plant lacking said mutated SP gene.

It is another object of the present invention to disclose the method as defined in any of the above, wherein said modified plant exhibits improved earliness as compared to a corresponding control Legume plant lacking said mutated SP gene.

It is another object of the present invention to disclose the method as defined in any of the above, wherein said modified plant exhibits suppressed sympodial shoot termination as compared to a corresponding control Legume plant lacking said mutated SP gene.

It is another object of the present invention to disclose the method as defined in any of the above, wherein said modified plant exhibits similar sympodial shoot termination as compared to a corresponding control Legume plant lacking said mutated SP gene.

It is another object of the present invention to disclose the method as defined in any of the above, wherein said modified plant exhibits suppressed or reduced day-length sensitivity as compared to a corresponding control Legume plant lacking said mutated SP gene.

It is another object of the present invention to disclose a modified Legume plant, plant part or plant cell produced by the method as defined in any of the above, wherein said modified plant does not comprise a transgene.

It is another object of the present invention to disclose a plant part, plant cell, plant pod or plant seed of a modified Legume plant produced by the method as defined in any of the above.

It is another object of the present invention to disclose a tissue culture of regenerable cells, protoplasts or callus obtained from the modified Legume plant produced by the method as defined in any of the above.

It is another object of the present invention to disclose the method as defined in any of the above, wherein said at least one domestication trait is selected from the group consisting of reduced flowering time, earliness, synchronous flowering, reduced day-length sensitivity, determinant or semi-determinant architecture, early termination of sympodial cycling, earlier axillary shoot flowering, compact growth habit, reduced height, reduced number of sympodial units, adaptation to mechanical harvest, higher harvest index and any combination thereof.

It is another object of the present invention to disclose an isolated polynucleotide sequence comprising at least 75% identity to a Legume SELF PRUNING (SP) sequence selected from the group consisting of SEQ ID NO:1, SEQ ID NO:113, SEQ ID NO:239, SEQ ID NO:340, SEQ ID NO:464, SEQ ID NO:616, SEQ ID NO:796, SEQ ID NO:1025, SEQ ID NO:1098, SEQ ID NO:1180, SEQ ID NO:1290, SEQ ID NO:1409, SEQ ID NO:1529, SEQ ID NO:1640, SEQ ID NO:1749 and SEQ ID NO:1928.

It is another object of the present invention to disclose an isolated polypeptide sequence comprising at least 75% identity to a Legume SELF PRUNING (SP) sequence selected from the group consisting of SEQ ID NO:2, SEQ ID NO:114, SEQ ID NO:240, SEQ ID NO:341, SEQ ID NO:465, SEQ ID NO:617, SEQ ID NO:797, SEQ ID NO:1026, SEQ ID NO:1099, SEQ ID NO:1181, SEQ ID NO:1291, SEQ ID NO:1410, SEQ ID NO:1530, SEQ ID NO:1641, SEQ ID NO:1750 and SEQ ID NO:1929.

It is another object of the present invention to disclose an isolated nucleotide sequence comprising least 75% sequence identity to a Legume SELF PRUNING (SP)-targeted gRNA sequence selected from the group consisting of SEQ ID NO:3-112, SEQ ID NO:115-238, SEQ ID NO:241-339, SEQ ID NO:342-463, SEQ ID NO:466-615, SEQ ID NO:618-795, SEQ ID NO:798-1024, SEQ ID NO:1027-1097, SEQ ID NO:1100-1179, SEQ ID NO:1182-1289, SEQ ID NO:1292-1408, SEQ ID NO:1411-1528, SEQ ID NO:1531-1639, SEQ ID NO:1642-1748, SEQ ID NO:1751-1927 and SEQ ID NO:1930-2051.

It is another object of the present invention to disclose harvestable parts of a modified Legume plant as defined in any of the above, wherein said harvestable parts are preferably shoot biomass and/or seeds.

It is another object of the present invention to disclose products derived from a modified comprising at least 75% sequence identity to plant as defined in any of the above, and/or from harvestable parts of a modified Legume plant as defined in any of the above.

It is another object of the present invention to disclose use of a nucleic acid encoding a polypeptide comprising at least 75% sequence identity to the sequence as defined in SEQ ID NO:2, SEQ ID NO:114, SEQ ID NO:240, SEQ ID NO:341, SEQ ID NO:465, SEQ ID NO:617, SEQ ID NO:797, SEQ ID NO:1026, SEQ ID NO:1099, SEQ ID NO:1181, SEQ ID NO:1291, SEQ ID NO:1410, SEQ ID NO:1530, SEQ ID NO:1641, SEQ ID NO:1750 and SEQ ID NO:1929, in enhancing yield and/or domestication, in Legume plants, relative to control plants.

### DETAILED DESCRIPTION OF THE PREFERRED EMBODIMENTS

In the following detailed description of the preferred embodiments, reference is made to the accompanying drawings that form a part hereof, and in which are shown by way of illustration specific embodiments in which the invention may be practiced. It is understood that other embodiments may be utilized and structural changes may be made without departing from the scope of the present invention. The present invention may be practiced according to the claims without some or all of these specific details. For the purpose of clarity, technical material that is known in the technical fields related to the invention has not been described in detail so that the present invention is not unnecessarily obscured.

The present invention provides a modified Legume plant exhibiting at least one improved domestication trait compared with a corresponding control or wild type Legume, wherein said modified Legume plant is a modified Peanut plant comprising at least one mutated Peanut (Arachis hypogaea) SP gene.

According to further aspects, the present invention provides a modified Legume plant exhibiting at least one improved domestication trait as compared to a corresponding control Legume plant, wherein said modified plant comprises a mutated SELF PRUNING (SP) Peanut (Arachis hypogaea) gene.

According to a further embodiment of the present invention, the Peanut (Arachis hypogaea) SP gene is selected from AhSP1-AhSP9 comprising a nucleic acid sequence with at least 75% sequence identity to a sequence selected from SEQ ID NO:1025, SEQ ID NO:1098, SEQ ID NO:1180, SEQ ID NO:1290, SEQ ID NO:1409, SEQ ID NO:1529, SEQ ID NO:1640, SEQ ID NO:1749 and SEQ ID NO:1928, respectively, or a functional variant thereof and any combination thereof.

The present invention further provides methods for producing the aforementioned modified Legume plant using genome editing or other genome modification techniques.

The solution proposed by the current invention is using genome editing such as the CRISPR/Cas system in order to create cultivated Legume plants with improved yield and more specifically with determinate growth habit. Breeding using genome editing allows a precise and significantly shorter breeding process in order to achieve these goals with a much higher success rate. Thus genome editing, has the potential to generate improved varieties faster and at a lower cost.

It is further noted that using genome editing is considered as non GMO by the Israeli regulator, and in the US, the USDA has already classified a dozen of genome edited plants as non-regulated and non GMO (https://www.usda.gov/media/press-releases/ 2018/03/28/secretary-perdue-issues-usda-statement-plant-breeding-innovation).

Legal limitations and outdated breeding techniques significantly hamper the efforts of generating new and improved Legume varieties adapted for intensive and advanced agriculture.

The present invention provides Legume plants with improved domestication traits such as plant architecture and plant habit adaptation. The current invention discloses the generation of non-transgenic Legume plants with improved yield traits, using the genome editing technology, e.g., the CRISPR/Cas9 highly precise tool. The generated mutations can be introduced into elite or locally adapted Legume lines rapidly, with relatively minimal effort and investment.

Genome editing is an efficient and useful tool for increasing crop productivity, and there is particular interest in advancing manipulation of domestication genes in

Legumes wild species, which often have undesirable characteristics.

Genome-editing technologies, such as the Clustered Regularly Interspaced Short Palindromic Repeats (CRISPR)-CRISPR-associated protein-9 nuclease (Cas9) (CRISPR-Cas9) provide opportunities to address these deficiencies, with the aims of increasing quality and yield, improve adaptation and expand geographical ranges of cultivation.

A major obstacle for CRISPR-Cas9 plant genome editing is lack of efficient tissue culture and transformation methodologies. The present invention achieves these aims and surprisingly provides transformed and regenerated Legume plants with modified desirable domestication genes.

To that end, guide RNAs (gRNAs) were designed for each of the target genes identified in Legumes to induce mutations in SP and genes through genome editing.

As used herein the term “about” denotes ±25% of the defined amount or measure or value.

As used herein the term “similar” denotes a correspondence or resemblance range of about ±20%, particularly ±15%, more particularly about ±10% and even more particularly about ±5%.

As used herein the term “corresponding” generally means similar, analogous, like, alike, akin, parallel, identical, resembling or comparable. In further aspects it means having or participating in the same relationship (such as type or degree, or species, kind, position, correspondence, function). It further means related or accompanying. In some embodiments of the present invention refers to plants of the same Legume species or strain or variety or to sibling plant, or one or more individuals having one or both parents in common.

A “plant” as used herein refers to any plant at any stage of development, particularly a seed plant. The term “plant” includes the whole plant or any parts or derivatives thereof, such as plant cells, seeds, plant protoplasts, plant cell tissue culture from which tomato plants can be regenerated, plant callus or calli, meristematic cells, microspores, embryos, immature embryos, pollen, ovules, anthers, fruit, flowers, leaves, cotyledons, pistil, seeds, seed coat, roots, root tips and the like.

The term “plant cell” used herein refers to a structural and physiological unit of a plant, comprising a protoplast and a cell wall. The plant cell may be in a form of an isolated single cell or a cultured cell.

The term “plant cell culture” as used herein means cultures of plant units such as, for example, protoplasts, regenerable cells, cell culture, cells, cells in plant tissues, pollen, pollen tubes, ovules, embryo sacs, zygotes and embryos at various stages of development, leaves, roots, root tips, anthers, meristematic cells, microspores, flowers, cotyledons, pistil, fruit, seeds, seed coat or any combination thereof.

The term “plant material” or “plant part” used herein refers to leaves, stems, roots, root tips, flowers or flower parts, fruits, pollen, egg cells, zygotes, seeds, seed coat, cuttings, cell or tissue cultures, or any other part or product of a plant or a combination thereof.

A “plant organ” as used herein means a distinct and visibly structured and differentiated part of a plant such as, but not limited to a root, stem, leaf, flower, flower bud, or embryo.

The term “Plant tissue” as used herein means a group of plant cells organized into a structural and functional unit. Any tissue of a plant in planta or in culture is included. This term includes, but is not limited to, plant organs, plant seeds, tissue culture, protoplasts, meristematic cells, calli and any group of plant cells organized into structural and/or functional units. The use of this term in conjunction with, or in the absence of, any specific type of plant tissue as listed above or otherwise embraced by this definition is not intended to be exclusive of any other type of plant tissue.

As used herein, the term “progeny” or “progenies” refers in a non-limiting manner to offspring or descendant plants. According to certain embodiments, the term “progeny” or “progenies” refers to plants developed or grown or produced from the disclosed or deposited seeds as detailed inter alia. The grown plants preferably have the desired traits of the disclosed or deposited seeds, i.e. loss of function mutation in at least one Peanut SP gene.

The term “Legume” refers hereinafter to a plant in the family Fabaceae (or Leguminosae), or the fruit or seed of such a plant. The legume family consists of plants that produce a pod with seeds inside.

The term “SELF-PRUNING” or “SP” in the context of the present invention refers to a gene which encodes a flowering repressor that modulates sympodial growth. It is herein shown that mutations in the SP orthologue cause an acceleration of sympodial cycling and shoot termination. It is further acknowledged that the SELF PRUNING (SP) gene controls the regularity of the vegetative-reproductive switch along the compound shoot of, for example, tomato, and thus conditions the ‘determinate’ (sp/sp) and ‘indeterminate’ (SP) growth habits of the plant. SP is a developmental regulator which is considered as similar to CENTRORADIALIS (CEN) from Antirrhinum and TERMINAL FLOWER 1 (TFL1) and FLOWERING LOCUS T (FT) from Arabidopsis.

The present invention discloses that SP is a member of a gene family in Legumes composed of at least 23 genes.

According to certain aspects of the present invention, the Legumes SP genes include Peanut SP genes.

The Peanut (Arachis hypogaea) SP gene is selected from AhSP1-AhSP9 comprising a genomic sequence as set forth in SEQ ID NO:1025, SEQ ID NO:1098, SEQ ID NO:1180, SEQ ID NO:1290, SEQ ID NO:1409, SEQ ID NO:1529, SEQ ID NO:1640, SEQ ID NO:1749 and SEQ ID NO:1928, respectively, and amino acid or polypeptide sequence as set forth in SEQ ID NO:1026, SEQ ID NO:1099, SEQ ID NO:1181, SEQ ID NO:1291, SEQ ID NO:1410, SEQ ID NO:1530, SEQ ID NO:1641, SEQ ID NO:1750 and SEQ ID NO:1929, respectively.

According to main aspects of the present invention, genome editing-targeted mutation in at least one of the aforementioned Peanut SP genes, which reduces the functional expression of the gene, affect the plant sympodial growth habit which plays a key role in determining plant architecture.

As used herein the term “genetic modification” refers hereinafter to genetic manipulation or modulation, which is the direct manipulation of an organism's genes using biotechnology. It also refers to a set of technologies used to change the genetic makeup of cells, including the transfer of genes within and across species, targeted mutagenesis and genome editing technologies to produce improved organisms. According to main embodiments of the present invention, modified Legume plants with improved domestication traits are generated using genome editing mechanism. This technique enables to achieve in planta modification of specific genes that relate to and/or control the flowering time and plant architecture in Legumes. The modification of the genes is aimed to result in modulated expression (preferably silencing) of the targeted genes, as compared to plants control lacking the generated modification.

The term “genome editing”, or “genome/genetic modification” or “genome engineering” or “gene editing” generally refers hereinafter to a type of genetic engineering in which DNA is inserted, deleted, modified or replaced in the genome of a living organism. Unlike previous genetic engineering techniques that randomly insert genetic material into a host genome, genome editing targets the insertions to site specific locations (e.g. specific genomic locus or loci).

It is within the scope of the present invention that the common methods for such editing use engineered nucleases, or “molecular scissors”. These nucleases create site-specific double-strand breaks (DSBs) at desired locations in the genome. The induced double-strand breaks are repaired through nonhomologous end-joining (NHEJ) or homologous recombination (HR), resulting in targeted mutations ('edits'). Families of engineered nucleases used by the current invention include, but are not limited to: meganucleases, zinc finger nucleases (ZFNs), transcription activator-like effector-based nucleases (TALEN), and the clustered regularly interspaced short palindromic repeats (CRISPR/Cas9) system.

Reference is now made to exemplary genome editing terms used by the current disclosure:

Cas=CRISPR-associated genes

Cas9, Csn1=a CRISPR-associated protein containing two nuclease domains, that is programmed by small RNAs to cleave DNA

crRNA=CRISPR RNA

dCAS9=nuclease-deficient Cas9

DSB=Double-Stranded Break

gRNA=guide RNA

HDR=Homology-Directed Repair

HNH=an endonuclease domain named for characteristic histidine and asparagine residues

Indel=insertion and/or deletion

NHEJ=Non-Homologous End Joining

PAM=Protospacer-Adjacent Motif

RuvC=an endonuclease domain named for an E. coli protein involved in DNA repair

sgRNA=single guide RNA

tracrRNA, trRNA=trans-activating crRNA

TALEN=Transcription-Activator Like Effector Nuclease

ZFN=Zinc-Finger Nuclease

According to specific aspects of the present invention, the CRISPR (Clustered Regularly Interspaced Short Palindromic Repeats) and CRISPR-associated (Cas) genes are used for the first time for generating genome modification in targeted genes in Legume plants. It is herein acknowledged that the functions of CRISPR (Clustered Regularly Interspaced Short Palindromic Repeats) and CRISPR-associated (Cas) genes are essential in adaptive immunity in selected bacteria and archaea, enabling the organisms to respond to and eliminate invading genetic material. These repeats were initially discovered in the 1980s in E. coli. Without wishing to be bound by theory, reference is now made to a type of CRISPR mechanism, in which invading DNA from viruses or plasmids is cut into small fragments and incorporated into a CRISPR locus comprising a series of short repeats (around 20 bps). The loci are transcribed, and transcripts are then processed to generate small RNAs (crRNA, namely CRISPR RNA), which are used to guide effector endonucleases that target invading DNA based on sequence complementarity.

According to further aspects of the invention, Cas protein, such as Cas9 (also known as Csn1) is required for gene silencing. Cas9 participates in the processing of crRNAs, and is responsible for the destruction of the target DNA. Cas9′s function in both of these steps relies on the presence of two nuclease domains, a RuvC-like nuclease domain located at the amino terminus, and a HNH-like nuclease domain that resides in the mid-region of the protein. To achieve site-specific DNA recognition and cleavage, Cas9 is complexed with both a crRNA and a separate trans-activating crRNA (tracrRNA or trRNA), that is partially complementary to the crRNA. The tracrRNA is required for crRNA maturation from a primary transcript encoding multiple pre-crRNAs. This occurs in the presence of RNase III and Cas9.

Without wishing to be bound by theory, it is herein acknowledged that during the destruction of target DNA, the HNH and RuvC-like nuclease domains cut both DNA strands, generating double-stranded breaks (DSBs) at sites defined by a 20-nucleotide target sequence within an associated crRNA transcript. The HNH domain cleaves the complementary strand, while the RuvC domain cleaves the noncomplementary strand.

It is further noted that the double-stranded endonuclease activity of Cas9 also requires that a short conserved sequence, (2-5 nts) known as protospacer-associated motif (PAM), follows immediately 3′- of the crRNA complementary sequence.

According to further aspects of the invention, a two-component system may be used by the current invention, combining trRNA and crRNA into a single synthetic single guide RNA (sgRNA) for guiding targeted gene alterations.

It is further within the scope that Cas9 nuclease variants include wild-type Cas9, Cas9D10A and nuclease-deficient Cas9 (dCas9).

Reference is now made to an example of CRISPR/Cas9 mechanism of action as depicted by Xie, Kabin, and Yinong Yang. “RNA-guided genome editing in plants using a CRISPR-Cas system.” Molecular plant 6.6 (2013): 1975-1983, incorporated herein by reference. As shown in this scientific publication, the Cas9 endonuclease forms a complex with a chimeric RNA (called guide RNA or gRNA), replacing the crRNA-transcrRNA heteroduplex, and the gRNA could be programmed to target specific sites. The gRNA-Cas9 should comprise at least 15-base-pairing (gRNA seed region) without mismatch between the 5′-end of engineered gRNA and targeted genomic site, and an NGG motif (called protospacer-adjacent motif or PAM) that follows the base-pairing region in the complementary strand of the targeted DNA.

The term “meganucleases” as used herein refers hereinafter to endodeoxyribonucleases characterized by a large recognition site (double-stranded DNA sequences of 12 to 40 base pairs); as a result this site generally occurs only once in any given genome. Meganucleases are therefore considered to be the most specific naturally occurring restriction enzymes.

The term “protospacer adjacent motif” or “PAM” as used herein refers hereinafter to a 2-6 base pair DNA sequence immediately following the DNA sequence targeted by the Cas9 nuclease in the CRISPR bacterial adaptive immune system. PAM is a component of the invading virus or plasmid, but is not a component of the bacterial CRISPR locus. PAM is an essential targeting component which distinguishes bacterial self from non-self DNA, thereby preventing the CRISPR locus from being targeted and destroyed by nuclease.

The term “Next-generation sequencing” or “NGS” as used herein refers hereinafter to massively, parallel, high-throughput or deep sequencing technology platforms that perform sequencing of millions of small fragments of DNA in parallel. Bioinformatics analyses are used to piece together these fragments by mapping the individual reads to the reference genome.

The term “gene knockdown” as used herein refers hereinafter to an experimental technique by which the expression of one or more of an organism's genes is reduced. The reduction can occur through genetic modification, i.e. targeted genome editing or by treatment with a reagent such as a short DNA or RNA oligonucleotide that has a sequence complementary to either gene or an mRNA transcript. The reduced expression can be at the level of RNA and/or at the level of protein. It is within the scope of the present invention that the term gene knockdown also refers to a loss of function mutation, gene knockout or silencing mutation in which an organism's genes is made inoperative or nonfunctional.

The term “gene silencing” as used herein refers hereinafter to the regulation of gene expression in a cell to prevent the expression of a certain gene. Gene silencing can occur during either transcription or translation. In certain aspects of the invention, gene silencing is considered to have a similar meaning as gene knockdown. When genes are silenced, their expression is reduced. In contrast, when genes are knocked out, they are completely not expressed. Gene silencing may be considered a gene knockdown mechanism since the methods used to silence genes, such as RNAi, CRISPR, or siRNA, generally reduce the expression of a gene by at least 70% but do not completely eliminate it.

The term “loss of function mutation” as used herein refers to a type of mutation in which the altered gene product lacks the function of the wild-type gene. A synonyms of the term included within the scope of the present invention is null mutation.

The term “microRNAs” or “miRNAs” refers hereinafter to small non-coding RNAs that have been found in most of the eukaryotic organisms. They are involved in the regulation of gene expression at the post-transcriptional level in a sequence specific manner. MiRNAs are produced from their precursors by Dicer-dependent small RNA biogenesis pathway. MiRNAs are candidates for studying gene function using different RNA-based gene silencing techniques. For example, artificial miRNAs (amiRNAs) targeting one or several genes of interest is a potential tool in functional genomics.

The term “in planta” means in the context of the present invention within the plant or plant cells. More specifically, it means introducing CRISPR/Cas complex into plant material comprising a tissue culture of several cells, a whole plant, or into a single plant cell, without introducing a foreign gene or a mutated gene. It also used to describe conditions present in a non-laboratory environment (e.g. in vivo).

The term ‘sympodial growth’ as used herein refers to a type of bifurcating branching pattern where one branch develops more strongly than the other, resulting in the stronger branches forming the primary shoot and the weaker branches appearing laterally. A sympodium, also referred to as a sympode or pseudaxis, is the primary shoot, comprising the stronger branches, formed during sympodial growth. In some aspects of the present invention, sympodial growth occurs when the apical meristem is terminated and growth is continued by one or more lateral meristems, which repeat the process. The apical meristem may be consumed to make an inflorescence or other determinate structure, or it may be aborted.

It is further within the scope of the current invention that the shoot section between two successive inflorescences is called the ‘sympodium’, and the number of leaf nodes per sympodium is referred to as the ‘sympodial index’ (spi). The first termination event activates the ‘sympodial cycle’. In sympodial plants, the apparent main shoot consists of a reiterated array of ‘sympodial units’. A mutant sp gene accelerates the termination of sympodial units but does not change the sympodial habit. The result is a progressive reduction in the number of vegetative nodes between inflorescences in a pattern that depends on light intensity and genetic background.

The term “earliness” refers hereinafter to early flowering and/or rapid transition from the vegetative to reproductive stages, or reduced ‘time to initiation of flowering’ and more generally to earlier completion of the life-cycle.

Plants having an “early flowering time” as used herein are plants which start to flower earlier than control plants. Hence this term refers to plants that show an earlier start of flowering. Flowering time of plants can be assessed by counting the number of days (“time to flower”) between sowing and the emergence of a first inflorescence. The “flowering time” of a plant can be determined using any method known in the art.

The term ‘reduced flowering time’ as used herein refers to time to production of first inflorescence. Such a trait can be evaluated or measured, for example, with reference to the number of leaves produced prior to appearance of the first inflorescence.

The term ‘harvest index’ can be herein defined as the total yield per plant weight.

The term ‘day length’ or ‘day length sensitivity’ as used in the context of the present invention generally refers to photoperiodism, which is the physiological reaction of organisms to the length of day or night. Photoperiodism can also be defined as the developmental responses of plants to the relative lengths of light and dark periods. Plants are classified under three groups according to the photoperiods: short-day plants, long-day plants, and day-neutral plants. Photoperiodism affects flowering by inducing the shoot to produce floral buds instead of leaves and lateral buds. It is within the scope of the present invention that Legumes are included within the short-day facultative plants. The Legume plants of the present invention are genetically modified so as to exhibit loss of day-length sensitivity, which is a highly desirable agronomical trait enabling enhanced yield of the cultivated crop.

The term ‘determinate’ or ‘determinate growth’ as used herein refers to plant growth in which the main stem ends in an inflorescence or other reproductive structure (e.g. a bud) and stops continuing to elongate indefinitely with only branches from the main stem having further and similarly restricted growth. It also refers to growth characterized by sequential flowering from the central or uppermost bud to the lateral or basal buds. It further means naturally self-limited growth, resulting in a plant of a definite maximum size.

The term ‘indeterminate’ or ‘indeterminate growth’ as used herein refers to plant growth in which the main stem continues to elongate indefinitely without being limited by a terminal inflorescence or other reproductive structure. It also refers to growth characterized by sequential flowering from the lateral or basal buds to the central or uppermost buds.

It is within the scope of the present invention that ‘yield related traits’ comprise one or more of early flowering time, yield, biomass, seed yield, early vigour, greenness index, increased growth rate and improved agronomic traits (such as improved plant architecture, i.e. determinate growth habit and enhanced nutritional value).

The term “yield” in general means a measurable produce of economic value, typically related to a specified crop, to an area, and to a period of time. Individual plant parts directly contribute to yield based on their number, size and/or weight, or the actual yield is the yield per square meter for a crop and year, which is determined by dividing total production (includes both harvested and appraised production) by planted square meters. The terms “yield” of a plant and “plant yield” are used interchangeably herein and are meant to refer to vegetative biomass such as root and/or shoot biomass, to reproductive organs, and/or to propagules such as seeds of that plant.

The terms “increase”, “improve” or “enhance” are interchangeable and shall mean in the sense of the application at least a 3%, 4%, 5%, 6%, 7%, 8%, 9% or 10%, preferably at least 15% or 20%, more preferably 258, 308, 35% or 40% more yield, growth or any other agronomic trait such as domestication trait or plant architecture, in comparison to control plants as defined herein.

Increased seed yield may be defined as one or more of the following: (a) an increase in seed biomass (total seed weight) which may be on an individual seed basis and/or per plant and/or per square meter; (b) increased number of flowers per plant; (c) increased number of seeds; and (d) increased harvest index, which is expressed as a ratio of the yield of harvestable parts, such as seeds, divided by the biomass of aboveground plant parts.

An increase in seed yield may also be manifested as an increase in seed size and/or seed volume.

The term “biomass” as used herein is intended to refer to the total weight of a plant. Within the definition of biomass, a distinction may be made between the biomass of one or more parts of a plant, which may include: aboveground (harvestable) parts such as but not limited to shoot biomass, seed biomass, leaf biomass, etc. and/or (harvestable) parts below ground, such as but not limited to root biomass, etc., and/or vegetative biomass such as root biomass, shoot biomass, etc., and/or reproductive organs, and/or propagules such as seed.

Control plant(s) within the scope of the present invention include corresponding wild type plants or corresponding naturally occurring plants or corresponding plants lacking the edited or mutated gene of interest or the specific generated mutation. The choice of suitable control plants is a routine part of an experimental setup and may include corresponding wild type plants or corresponding plants without the gene of interest. The control plant is typically of the same plant species or the same genetic background or even of the same variety as the plant to be assessed. The control plant of the plant to be assessed may also be plant individuals missing the transgene or modified/edited gene. A “control plant” or a “wild type” plant as used herein refers not only to whole plants, but also to plant parts, including seeds and seed parts.

The term “orthologue” as used herein refers hereinafter to one of two or more homologous gene sequences found in different species.

The term “functional variant” or “functional variant of a nucleic acid or amino acid sequence” as used herein refers to a sequence or part of a sequence which retains the biological function of the full non-variant allele (e.g. Peanut SP genes) and hence has the activity of SP expressed gene or protein. A functional variant also comprises a variant of the gene of interest encoding a polypeptide which has sequence alterations that do not affect function of the resulting protein, for example, in non-conserved residues. Also encompassed is a variant that is substantially identical, i.e. has only some sequence variations, for example, in non-conserved residues, to the wild type nucleic acid or amino acid sequences of the alleles as shown herein, and is biologically active.

The term ‘resistant’ or ‘less sensitive’ as used herein refers to plant growth in which the plant either expresses no symptoms or less symptoms of a disease conferred by an associated pathogen in comparison to a plant that is sensitive to such pathogen.

The term “variety” or “cultivar” used herein means a group of similar plants that by structural features and performance can be identified from other varieties within the same species.

The term “allele” used herein means any of one or more alternative or variant forms of a gene or a genetic unit at a particular locus, all of which alleles relate to one trait or characteristic at a specific locus. In a diploid cell of an organism, alleles of a given gene are located at a specific location, or locus (loci plural) on a chromosome. Alternative or variant forms of alleles may be the result of single nucleotide polymorphisms, insertions, inversions, translocations or deletions, or the consequence of gene regulation caused by, for example, by chemical or structural modification, transcription regulation or post-translational modification/regulation. An allele associated a with qualitative trait may comprise alternative or variant forms of various genetic units including those that are identical or associated with a single gene or multiple genes or their products or even a gene disrupting or controlled by a genetic factor contributing to the phenotype represented by the locus. According to further embodiments, the term “allele” designates any of one or more alternative forms of a gene at a particular locus. Heterozygous alleles are two different alleles at the same locus. Homozygous alleles are two identical alleles at a particular locus. A wild type allele is a naturally occurring allele. In the context of the current invention, the term allele refers to the 16 identified SP legumes genes, having the genomic nucleotide sequence as set forth in SEQ ID NO:1025, SEQ ID NO:1098, SEQ ID NO:1180, SEQ ID NO:1290, SEQ ID NO:1409, SEQ ID NO:1529, SEQ ID NO:1640, SEQ ID NO:1749 and SEQ ID NO:1928 for Peanut SP.

As used herein, the term “locus” (loci plural) means a specific place(s) or region(s) or a site(s) on a chromosome where for example a gene or genetic marker element or factor is found. In specific embodiments, such a genetic element is contributing to a trait.

As used herein, the term “homozygous” refers to a genetic condition or configuration existing when two identical or like alleles reside at a specific locus, but are positioned individually on corresponding pairs of homologous chromosomes in the cell of a diploid organism.

In specific embodiments, the Legume plants of the present invention comprise homozygous configuration of at least one of the mutated Peanut SP genes.

Conversely, as used herein, the term “heterozygous” means a genetic condition or configuration existing when two different or unlike alleles reside at a specific locus, but are positioned individually on corresponding pairs of homologous chromosomes in the cell of a diploid organism.

As used herein, the phrase “genetic marker” or “molecular marker” or “biomarker” refers to a feature in an individual's genome e.g., a nucleotide or a polynucleotide sequence that is associated with one or more loci or trait of interest In some embodiments, a genetic marker is polymorphic in a population of interest, or the locus occupied by the polymorphism, depending on context. Genetic markers or molecular markers include, for example, single nucleotide polymorphisms (SNPs), indels (i.e. insertions deletions), simple sequence repeats (SSRs), restriction fragment length polymorphisms (RFLPs), random amplified polymorphic DNAs (RAFDs), cleaved amplified polymorphic sequence (CAPS) markers, Diversity Arrays Technology (DArT) markers, and amplified fragment length polymorphisms (AFLPs) or combinations thereof, among many other examples such as the DNA sequence per se. Genetic markers can, for example, be used to locate genetic loci containing alleles on a chromosome that contribute to variability of phenotypic traits. The phrase “genetic marker” or “molecular marker” or “biomarker” can also refer to a polynucleotide sequence complementary or corresponding to a genomic sequence, such as a sequence of a nucleic acid used as a probe or primer.

As used herein, the term “germplasm” refers to the totality of the genotypes of a population or other group of individuals (e.g., a species). The term “germplasm” can also refer to plant material; e.g., a group of plants that act as a repository for various alleles. Such germplasm genotypes or populations include plant materials of proven genetic superiority; e.g., for a given environment or geographical area, and plant materials of unknown or unproven genetic value; that are not part of an established breeding population and that do not have a known relationship to a member of the established breeding population.

The terms “hybrid”, “hybrid plant” and “hybrid progeny” used herein refers to an individual produced from genetically different parents (e.g., a genetically heterozygous or mostly heterozygous individual).

As used herein, “sequence identity” or “identity” in the context of two nucleic acid or polypeptide sequences makes reference to the residues in the two sequences that are the same when aligned for maximum correspondence over a specified comparison window. When percentage of sequence identity is used in reference to proteins, it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. The term further refers hereinafter to the amount of characters which match exactly between two different sequences. Hereby, gaps are not counted and the measurement is relational to the shorter of the two sequences.

It is further within the scope that the terms “similarity” and “identity” additionally refer to local homology, identifying domains that are homologous or similar (in nucleotide and/or amino acid sequence). It is acknowledged that bioinformatics tools such as BLAST, SSEARCH, FASTA, and HMMER calculate local sequence alignments which identify the most similar region between two sequences. For domains that are found in different sequence contexts in different proteins, the alignment should be limited to the homologous domain, since the domain homology is providing the sequence similarity captured in the score. According to some aspects the term similarity or identity further includes a sequence motif, which is a nucleotide or amino-acid sequence pattern that is widespread and has, or is conjectured to have, a biological significance. Proteins may have a sequence motif and/or a structural motif, a motif formed by the three-dimensional arrangement of amino acids which may not be adjacent.

As used herein, the terms “nucleic acid”, “nucleic acid sequence”, “nucleotide”, “nucleic acid molecule” or “polynucleotide” are intended to include DNA molecules (e.g., CDNA or genomic DNA), RNA molecules (e.g., mRNA), natural occurring, mutated, synthetic DNA or RNA molecules, and analogs of the DNA or RNA generated using nucleotide analogs. It can be single-stranded or double-stranded. Such nucleic acids or polynucleotides include, but are not limited to, coding sequences of structural genes, anti-sense sequences, and non-coding regulatory sequences that do not encode mRNAs or protein products. These terms also encompass a gene. The term “gene”, “allele” or “gene sequence” is used broadly to refer to a DNA nucleic acid associated with a biological function. Thus, genes may include introns and exons as in the genomic sequence, or may comprise only a coding sequence as in cDNAs, in their gene expressed form, and/or may include cDNAs in combination with regulatory sequences. Thus, according to the various aspects of the invention, genomic DNA, CDNA or coding DNA may be used. In one embodiment, the nucleic acid is cDNA or coding DNA.

The terms “peptide”, “polypeptide” and “protein” are used interchangeably herein and refer to amino acids in a polymeric form of any length, linked together by peptide bonds.

According to other aspects of the invention, a “modified” or a “mutant” plant is a plant that has been altered compared to the naturally occurring wild type (WT) or control plant. Specifically, the endogenous nucleic acid sequences of each of the SP homologs in Legumes (nucleic acid sequences comprising at least 75% sequence identity to SEQ ID NO:1025, SEQ ID NO:1098, SEQ ID NO:1180, SEQ ID NO:1290, SEQ ID NO:1409, SEQ ID NO:1529, SEQ ID NO:1640, SEQ ID NO:1749 and SEQ ID NO:1928 for Peanut SP genes) have been altered compared to wild type sequences using mutagenesis and/or genome editing methods as described herein. This causes inactivation of the endogenous SP gene and thus disables SP function and/or expression. Such plants have an altered phenotype and show improved domestication traits such as determinant plant architecture, synchronous and/or early flowering and loss of day length sensitivity compared to corresponding wild type plants or control plants lacking the SP modification. Therefore, the improved domestication phenotype is conferred by the presence of at least one mutated endogenous Peanut SP gene in the Legumes plant genome which has been specifically targeted using genome editing technique.

According to further aspects of the present invention, the at least one improved domestication trait is not conferred by the presence of transgenes expressed in Legumes.

It is further within the scope of the current invention that sp mutations that down-regulate or disrupt functional expression of the wild-type SP gene sequence, may be recessive, such that they are complemented by expression of a wild-type sequence.

It is further noted that according to certain aspects of the present invention, a wild type Legume plant is a plant that does not have any mutant sp allele.

Main aspects invention of the involve targeted mutagenesis methods, specifically genome editing, and exclude embodiments that are solely based on generating plants by traditional breeding methods. In a further embodiment of the current invention, as explained herein, the improved domestication of at least one trait is not due to the presence of a transgene.

The inventors have generated mutant Legume lines with mutations inactivating at least one Peanut SP gene homoeoallele which confer heritable improved domestication trait(s). In this way no functional at least one Peanut SP protein is made. Thus, the invention relates to these mutant Legume lines and related methods.

It is further within the scope of the present invention that breeding Legume cultivars with mutated sp allele enables the mechanical harvest of the plant. According to a further aspect of the present invention, loss of SP function results in compact Legume plants with reduced height, reduced number of sympodial units and determinate growth when compared with corresponding WT Legumes.

According to a main aspect of the present invention, modifying Legumes shoot architecture by selection for mutations in florigen flowering pathway genes allowed major improvements in plant architecture and yield. In particular, a mutation in an antiflorigen SELFPRUNING (SP) gene (sp classic) provided compact ‘determinate’ growth that translated to a burst of flowers, thereby enabling largescale field production.

The work inter alia described has important implications. The results have shown that CRISPR/Cas9 can be used to create heritable mutations in florigen pathway family members that result in desirable phenotypic effects.

To edit multiple domestication genes simultaneously and stack the resulting allelic variants, one option is that several gRNAs can be assembled to edit several genes into one construct, by using the Csy4 multi-gRNA system. The construct is then transformed via an appropriate vector into several Legume accessions.

It is further within the scope of the current invention that Legume SP genes having genomic nucleotide sequence as set forth in SEQ ID NO:1025, SEQ ID NO:1098, SEQ ID NO:1180, SEQ ID NO:1290, SEQ ID NO:1409, SEQ ID NO:1529, SEQ ID NO:1640, SEQ ID NO:1749 and SEQ ID NO:1928 for Peanut, are silenced by genome editing. Several mutated alleles have been identified. Notably, the plants with mutated sp alleles were more compact than the wild type plants lacking the mutated allele.

The loss of function mutation may be a deletion or insertion (“indels”) with reference the wild type Peanut SP gene allele sequence. The deletion may comprise 1-20 or more nucleotides, for example 1, 2, 3, 4, 5, 6, 7, 8, 9, 10, 1, 12, 13, 14, 15, 16, 17, 18 or 20 nucleotides or more in one or more strand. The insertion may comprise 1-20 or more nucleotides, for example 1, 2, 3, 4, 5, 6, 7, 8, 9, 10, 1, 12, 13, 14, 15, 16, 17, 18 or 20 or more nucleotides in one or more strand.

The plant of the invention includes plants wherein the plant is heterozygous for the each of the mutations. In a preferred embodiment however, the plant is homozygous for the mutations. Progeny that is also homozygous can be generated from these plants according to methods known in the art.

It is further within the scope that variants of a particular Peanut SP gene nucleotide or amino acid encoded sequence according to the various aspects of the invention will have at least about 50%- 99%, for example at least 75%, for example at least 85%, 86%, 87%, 88%, 89%, 90%, 92%, 94%, 95%, 96%, 97%, 98% or 99% or more sequence identity to that particular non-variant Peanut SP gene nucleotide sequence as shown in SEQ ID NO:1025, SEQ ID NO:1098, SEQ ID NO:1180, SEQ ID NO:1290, SEQ ID NO:1409, SEQ ID NO:1529, SEQ ID NO:1640, SEQ ID NO:1749 and SEQ ID NO:1928 for peanut.

Also, the various aspects of the invention encompass not only a Peanut SP genes or any other aforementioned nucleic acid sequence or amino acid sequence, but also fragments thereof. By “fragment” it is intended to mean a portion of the nucleotide sequence or a portion of the amino acid sequence and hence of the protein encoded thereby. Fragments of a nucleotide sequence may encode protein fragments that retain the biological activity of the native protein, in this case improved domestication trait.

According to a further embodiment of the invention, the herein newly identified Legume SP (Peanut SP genes) have been targeted using the double sgRNA strategy.

According to further embodiments of the present invention, DNA introduction into the plant cells can be done by Agrobacterium infiltration, virus based plasmids for delivery of the genome editing molecules and mechanical insertion of DNA (PEG mediated DNA transformation, biolistics, etc.).

In addition, it is within the scope of the present invention that the Cas9 protein is directly inserted together with a gRNA (ribonucleoprotein—RNP's) in order to bypass the need for in vivo transcription and translation of the Cas9+gRNA plasmid in planta to achieve gene editing.

It is also possible to create a genome edited plant and use it as a rootstock. Then, the Cas protein and gRNA can be transported via the vasculature system to the top of the plant and create the genome editing event in the scion.

It is within the scope of the present invention that the usage of CRISPR/Cas system for the generation of Legume plants with at least one improved domestication trait, allows the modification of predetermined specific DNA sequences without introducing foreign DNA into the genome by GMO techniques. According to one embodiment of the present invention, this is achieved by combining the Cas nuclease (e.g. Cas9, Cpf1 and the like) with a predefined guide RNA molecule (gRNA). The gRNA is complementary to a specific DNA sequence targeted for editing in the plant genome and which guides the Cas nuclease to a specific nucleotide sequence. The predefined gene specific gRNA's are cloned into the same plasmid as the Cas gene and this plasmid is inserted into plant cells. Insertion of the aforementioned plasmid DNA can be done, but not limited to, using different delivery systems, biological and/or mechanical, e.g. Agrobacterium infiltration, virus based plasmids for delivery of the genome editing molecules and mechanical insertion of DNA (PEG mediated DNA transformation, biolistics, etc.).

It is further within the scope of the present invention that upon reaching the specific predetermined DNA sequence, the Cas9 nuclease cleaves both DNA strands to create double stranded breaks leaving blunt ends. This cleavage site is then repaired by the cellular non homologous end joining DNA repair mechanism resulting in insertions or deletions which eventually create a mutation at the cleavage site. For example, it is acknowledged that a deletion form of the mutation consists of at least 1 base pair deletion. As a result of this base pair deletion the gene coding sequence is disrupted and the translation of the encoded protein is compromised either by a premature stop codon or disruption of a functional or structural property of the protein. Thus DNA is cut by the Cas9 protein and re-assembled by the cell's DNA repair mechanism.

It is further within the scope that improved domestication traits in Legume plants is herein produced by generating gRNA with homology to a specific site of predetermined genes in the Legumes genome i.e. SP gene, sub cloning this gRNA into a plasmid containing the Cas9 gene, and insertion of the plasmid into the Legume plant cells. In this way site specific mutations in the SP and aforementioned genes are generated thus effectively creating non-active molecules, resulting in determinant growth habit of the genome edited plant.

According to one embodiment, the present invention provides a modified Legume plant exhibiting at least one improved domestication trait as compared to a corresponding control Legume plant, wherein said modified plant comprises a mutated SELF PRUNING (SP) Peanut (Arachis hypogaea) gene.

According to a further embodiment, the present invention discloses the modified Legume plant as defined in any of the above, wherein said Peanut (Arachis hypogaea) SP gene is selected from AhSP1-AhSP9 comprising a nucleic acid sequence with at least 75% sequence identity to a sequence selected from SEQ ID NO:1025, SEQ ID NO:1098, SEQ ID NO:1180, SEQ ID NO:1290, SEQ ID NO:1409, SEQ ID NO:1529, SEQ ID NO:1640, SEQ ID NO:1749 and SEQ ID NO:1928, respectively, or a functional variant thereof and any combination thereof.

According to a further embodiment, the present invention discloses the modified Legume plant as defined in any of the above, wherein said mutation is introduced using mutagenesis, small interfering RNA (siRNA), microRNA (miRNA), artificial miRNA (amiRNA), DNA introgression, endonucleases or any combination thereof.

According to a further embodiment, the present invention discloses the modified Legume plant as defined in any of the above, wherein said mutation is introduced using targeted genome modification.

According to a further embodiment, the present invention discloses the modified Legume plant as defined in any of the above, wherein said mutation is introduced using CRISPR (Clustered Regularly Interspaced Short Palindromic Repeats) and CRISPR-associated (Cas) gene (CRISPR/Cas), Transcription activator-like effector nuclease (TALEN), Zinc Finger Nuclease (ZFN), meganuclease or any combination thereof.

According to a further embodiment, the present invention discloses the modified Legume plant as defined in any of the above, wherein said Cas gene is selected from the group consisting of Cas9, Cas12, Cas13, Cas14, CasX, CasY, Csn1, Cpf1 and any combination thereof.

According to a further embodiment, the present invention discloses the modified Legume plant as defined in any of the above, wherein the mutated SP gene is a CRISPR/Cas9-induced heritable mutated allele.

According to a further embodiment, the present invention discloses the modified Legume plant as defined in any of the above, wherein said mutation is a missense mutation, nonsense mutation, insertion, deletion, indel, substitution or duplication.

According to a further embodiment, the present invention discloses the modified Legume plant as defined in any of the above, wherein the insertion or the deletion produces a gene comprising a frameshift.

According to a further embodiment, the present invention discloses the modified Legume plant as defined in any of the above, wherein said plant is homozygous for said at least one mutated SP gene.

According to a further embodiment, the present invention discloses the modified Legume plant as defined in any of the above, wherein said mutation is in the coding region of said gene, a mutation in the regulatory region of said gene, or an epigenetic factor.

According to a further embodiment, the present invention discloses the modified Legume plant as defined in any of the above, wherein said mutation is a silencing mutation, a knockdown mutation, a knockout mutation, a loss of function mutation or any combination thereof.

According to a further embodiment, the present invention discloses the modified Legume plant as defined in any of the above, wherein said mutation is generated in planta.

According to a further embodiment, the present invention discloses the modified Legume plant as defined in any of the above, wherein said mutation is generated in planta via introduction of a construct comprising (a) Cas DNA and gRNA sequence selected from the group consisting of SEQ ID NO:1027-1097, SEQ ID NO:1100-1179, SEQ ID NO:1182-1289, SEQ ID NO:1292-1408, SEQ ID NO:1411-1528, SEQ ID NO:1531-1639, SEQ ID NO:1642-1748, SEQ ID NO:1751-1927 and SEQ ID NO:1930-2051, for said at least one Peanut SP gene, and any combination thereof, or (b) a ribonucleoprotein (RNP) complex comprising Cas protein and gRNA sequence selected from the group consisting of SEQ ID NO:1027-1097, SEQ ID NO:1100-1179, SEQ ID NO:1182-1289, SEQ ID NO:1292-1408, SEQ ID NO:1411-1528, SEQ ID NO:1531-1639, SEQ ID NO:1642-1748, SEQ ID NO:1751-1927 and SEQ ID NO:1930-2051 for said at least one Peanut SP gene, and any combination thereof.

According to a further embodiment, the present invention discloses the modified Legume plant as defined in any of the above, wherein said mutation in said at least one Peanut SP gene is generated in planta via introduction of a construct comprising (a) Cas DNA and gRNA sequence selected from the group consisting of SEQ ID NO:1027-1097, SEQ ID NO:1100-1179, SEQ ID NO:1182-1289, SEQ ID NO:1292-1408, SEQ ID NO:1411-1528, SEQ ID NO:1531-1639, SEQ ID NO:1642-1748, SEQ ID NO:1751-1927 and SEQ ID NO:1930-2051 for Peanut SEQ ID

NO:1025, SEQ ID NO:1098, SEQ ID NO:1180, SEQ ID NO:1290, SEQ ID NO:1409, SEQ ID NO:1529, SEQ ID NO:1640, SEQ ID NO:1749 and SEQ ID NO:1928, respectively, and any combination thereof, or (b) a ribonucleoprotein (RNP) complex comprising Cas protein and gRNA sequence selected from the group consisting of SEQ ID NO:1027-1097, SEQ ID NO:1100-1179, SEQ ID NO:1182-1289, SEQ ID NO:1292-1408, SEQ ID NO:1411-1528, SEQ ID NO:1531-1639, SEQ ID NO:1642-1748, SEQ ID NO:1751-1927 and SEQ ID NO:1930-2051 for Peanut SEQ ID NO:1025, SEQ ID NO:1098, SEQ ID NO:1180, SEQ ID NO:1290, SEQ ID NO:1409, SEQ ID NO:1529, SEQ ID NO:1640, SEQ ID NO:1749 and SEQ ID NO:1928, respectively, and any combination thereof.

According to a further embodiment, the present invention discloses the modified Legume plant as defined in any of the above, wherein said gRNA sequence comprises a 3′ NGG Protospacer Adjacent Motif (PAM).

According to a further embodiment, the present invention discloses the modified Legume plant as defined in any of the above, wherein said construct is introduced into the plant cells via Agrobacterium infiltration, virus-based plasmids for delivery of genome editing molecules, or mechanical insertion such as polyethylene glycol (PEG) mediated DNA transformation, electroporation or gene gun biolistics.

According to a further embodiment, the present invention discloses the modified Legume plant as defined in any of the above, wherein said mutation confers reduced expression of said at least one SP gene.

According to a further embodiment, the present invention discloses the modified Legume plant as defined in any of the above, wherein said modified plant has decreased expression levels of said SP gene.

According to a further embodiment, the present invention discloses the modified Legume plant as defined in any of the above, wherein the sequence of said expressed SP gene is selected from the group consisting of: at least 75% identity to any one of Peanut polypeptide SEQ ID NO:1026, SEQ ID NO:1099, SEQ ID NO:1181, SEQ ID NO:1291, SEQ ID NO:1410, SEQ ID NO:1530, SEQ ID NO:1641, SEQ ID NO:1750 and SEQ ID NO:1929, or a functional variant thereof.

According to a further embodiment, the present invention discloses the modified Legume plant as defined in any of the above, wherein said SP gene encodes a polypeptide sequence selected from the group consisting of: at least 75% identity to any one of Peanut polypeptide SEQ ID NO:1026, SEQ ID NO:1099, SEQ ID NO:1181, SEQ ID NO:1291, SEQ ID NO:1410, SEQ ID NO:1530, SEQ ID NO:1641, SEQ ID NO:1750 and SEQ ID NO:1929, or a functional variant thereof.

According to a further embodiment, the present invention discloses the modified Legume plant as defined in any of the above, wherein said modified plant is semi-determinant.

According to a further embodiment, the present invention discloses the modified Legume plant as defined in any of the above, wherein said modified plant has determinant growth habit.

According to a further embodiment, the present invention discloses the modified Legume plant as defined in any of the above, wherein said modified plant flowers earlier than a corresponding control Legume plant lacking said mutated SP gene.

According to a further embodiment, the present invention discloses the modified Legume plant as defined in any of the above, wherein said modified plant exhibits improved earliness as compared to a corresponding control Legume plant lacking said mutated SP gene.

According to a further embodiment, the present invention discloses the modified Legume plant as defined in any of the above, wherein said modified plant exhibits suppressed and/or similar sympodial shoot termination as compared to a corresponding control Legume plant lacking said mutated SP gene.

According to a further embodiment, the present invention discloses the modified Legume plant as defined in any of the above, wherein said domestication trait is selected from the group consisting of reduced flowering time, earliness, synchronous flowering, reduced day-length sensitivity, determinant or semi-determinant architecture, early termination of sympodial cycling, earlier axillary shoot flowering, compact growth habit, reduced height, reduced number of sympodial units, adaptation to mechanical harvest, higher harvest index and any combination thereof.

According to a further embodiment, the present invention discloses a modified Legume plant, plant part, plant tissue or plant cell as defined in any of the above, wherein said plant does not comprise a transgene.

According to a further embodiment, the present invention discloses a plant part, plant cell, plant pod or plant seed of a modified Legume plant as defined in any of the above.

According to a further embodiment, the present invention discloses a tissue culture of regenerable cells, protoplasts or callus obtained from the modified Legume plant as defined in any of the above.

According to a further embodiment, the present invention discloses a method for producing a modified Legume plant exhibiting at least one improved domestication trait compared with a corresponding control Legume, said method comprises steps of genetically modifying at least one Legume SELF PRUNING (SP) Peanut (Arachis hypogaea) gene, the resultant mutated SP gene has reduced expression level.

According to a further embodiment, the present invention discloses the method as defined in any of the above, wherein said method comprises steps of genetically modifying the at least one Legume SP gene using targeted genome editing introducing a loss of function mutation in the at least one Peanut (Arachis hypogaea) SP gene.

According to a further embodiment, the present invention discloses the method as defined in any of the above, wherein said Peanut (Arachis hypogaea) SP gene is selected from AhSP1-AhSP9 comprising a nucleic acid sequence with at least 75% sequence identity to a sequence selected from SEQ ID NO:1025, SEQ ID NO:1098, SEQ ID NO:1180, SEQ ID NO:1290, SEQ ID NO:1409, SEQ ID NO:1529, SEQ ID NO:1640, SEQ ID NO:1749 and SEQ ID NO:1928, or a functional variant thereof and any combination thereof.

According to a further embodiment, the present invention discloses the method as defined in any of the above, wherein said method comprises steps of: (a) identifying at least one Legume SP gene in a predetermined Legume plant; (b) synthetizing at least one guide RNA (gRNA) comprising a nucleotide sequence complementary to said at least one identified Legume SP gene; (c) transforming the predetermined Legume plant cells with a construct comprising (i) Cas nucleotide sequence operably linked to said at least one gRNA, or (ii) a ribonucleoprotein (RNP) complex comprising Cas protein and said at least one gRNA; (d) screening the genome of said transformed predetermined Legume plant cells for induced targeted loss of function mutation in said at least one Legume SP gene; (e) regenerating Legume plants carrying said loss of function mutation in at least one of said Legume SP gene; and (f) screening said regenerated plants for a Legume plant with improved domestication trait.

According to a further embodiment, the present invention discloses the method as defined in any of the above, wherein said step of screening the genome of said transformed plant cells for induced targeted loss of function mutation further comprises steps of obtaining a nucleic acid sample of said transformed plant and performing a nucleic acid amplification and optionally restriction enzyme digestion to detect a mutation in said at least one of said Legume SP gene.

According to a further embodiment, the present invention discloses the method as defined in any of the above, wherein said SP gene is selected from the group consisting of Peanut SP gene comprising a sequence having at least 75% identity to a sequence selected from SEQ ID NO:1025, SEQ ID NO:1098, SEQ ID NO:1180, SEQ ID NO:1290, SEQ ID NO:1409, SEQ ID NO:1529, SEQ ID NO:1640, SEQ ID NO:1749 and SEQ ID NO:1928, or a functional variant thereof and any combination thereof.

According to a further embodiment, the present invention discloses the method as defined in any of the above, wherein said mutation is introduced using mutagenesis, small interfering RNA (siRNA), microRNA (miRNA), artificial miRNA (amiRNA), DNA introgression, endonucleases or any combination thereof.

According to a further embodiment, the present invention discloses the method as defined in any of the above, wherein said mutation is introduced using targeted genome modification.

According to a further embodiment, the present invention discloses the method as defined in any of the above, wherein said mutation is introduced using CRISPR (Clustered Regularly Interspaced Short Palindromic Repeats) and CRISPR-associated (Cas) gene (CRISPR/Cas), Transcription activator-like effector nuclease (TALEN), Zinc Finger Nuclease (ZEN), meganuclease or any combination thereof.

According to a further embodiment, the present invention discloses the method as defined in any of the above, wherein said Cas gene is selected from the group consisting of Cas9, Cas12, Cas13, Cas14, CasX, CasY, Csn1, Cpf1 and any combination thereof.

According to a further embodiment, the present invention discloses the method as defined in any of the above, wherein the mutated SP gene is a CRISPR/Cas9-induced heritable mutated allele.

According to a further embodiment, the present invention discloses the method as defined in any of the above, wherein said mutation is a missense mutation, nonsense mutation, insertion, deletion, indel, substitution or duplication.

According to a further embodiment, the present invention discloses the method as defined in any of the above, wherein the insertion or the deletion produces a gene comprising a frameshift.

According to a further embodiment, the present invention discloses the method as defined in any of the above, wherein said plant is homozygous for said at least one Legume SP gene.

According to a further embodiment, the present invention discloses the method as defined in any of the above, wherein said mutation is in the coding region of said gene, a mutation in the regulatory region of said gene, or an epigenetic factor.

According to a further embodiment, the present invention discloses the method as defined in any of the above, wherein said mutation is a silencing mutation, a knockdown mutation, a knockout mutation, a loss of function mutation or any combination thereof.

According to a further embodiment, the present invention discloses the method as defined in any of the above, wherein said mutation is generated in planta.

According to a further embodiment, the present invention discloses the method as defined in any of the above, wherein said mutation is generated in planta via introduction of a construct comprising (a) Cas DNA and gRNA sequence selected from the group consisting of SEQ ID NO:1027-1097, SEQ ID NO:1100-1179, SEQ ID NO:1182-1289, SEQ ID NO:1292-1408, SEQ ID NO:1411-1528, SEQ ID NO:1531-1639, SEQ ID NO:1642-1748, SEQ ID NO:1751-1927 and SEQ ID NO:1930-2051, for said at least one Peanut SP gene, and any combination thereof, or (b) a ribonucleoprotein (RNP) complex comprising Cas protein and gRNA sequence selected from the group consisting of SEQ ID NO:1027-1097, SEQ ID NO:1100-1179, SEQ ID NO:1182-1289, SEQ ID NO:1292-1408, SEQ ID NO:1411-1528, SEQ ID NO:1531-1639, SEQ ID NO:1642-1748, SEQ ID NO:1751-1927 and SEQ ID NO:1930-2051, for said at least one Peanut SP gene, and any combination thereof.

According to a further embodiment, the present invention discloses the method as defined in any of the above, wherein said mutation in said Peanut SP genes is generated in planta via introduction of a construct comprising (a) Cas DNA and gRNA sequence selected from the group consisting of SEQ ID NO:1027-1097, SEQ ID NO:1100-1179, SEQ ID NO:1182-1289, SEQ ID NO:1292-1408, SEQ ID NO:1411-1528, SEQ ID NO:1531-1639, SEQ ID NO:1642-1748, SEQ ID NO:1751-1927 and SEQ ID NO:1930-2051 for Peanut SEQ ID NO:1025, SEQ ID NO:1098, SEQ ID NO:1180, SEQ ID NO:1290, SEQ ID NO:1409, SEQ ID NO:1529, SEQ ID NO:1640, SEQ ID NO:1749 and SEQ ID NO:1928, respectively, or (b) a ribonucleoprotein (RNP) complex comprising Cas protein and gRNA sequence selected from the group consisting of SEQ ID NO:1027-1097, SEQ ID NO:1100-1179, SEQ ID NO:1182-1289, SEQ ID NO:1292-1408, SEQ ID NO:1411-1528, SEQ ID NO:1531-1639, SEQ ID NO:1642-1748, SEQ ID NO:1751-1927 and SEQ ID NO:1930-2051 for Peanut SEQ ID NO:1025, SEQ ID NO:1098, SEQ ID NO:1180, SEQ ID NO:1290, SEQ ID NO:1409, SEQ ID NO:1529, SEQ ID NO:1640, SEQ ID NO:1749 and SEQ ID NO:1928, respectively.

According to a further embodiment, the present invention discloses the method as defined in any of the above, wherein said gRNA sequence comprises a 3′ NGG Protospacer Adjacent Motif (PAM).

According to a further embodiment, the present invention discloses the method as defined in any of the above, wherein said construct is introduced into the plant cells via Agrobacterium infiltration, virus based plasmids for delivery of the genome editing molecules or mechanical insertion such as polyethylene glycol (PEG) mediated DNA transformation, electroporation or gene gun biolistics.

According to a further embodiment, the present invention discloses the method as defined in any of the above, wherein said modified plant has decreased expression levels of at least one of said Legume SELF PRUNING (SP) gene.

According to a further embodiment, the present invention discloses the method as defined in any of the above, wherein said mutation confers reduced expression of said at least one SP gene.

According to a further embodiment, the present invention discloses the method as defined in any of the above, wherein the sequence of said expressed Legume SELF PRUNING (SP) gene is selected from the group consisting of: at least 75% identity to any one of Peanut polypeptide SEQ ID NO:1026, SEQ ID NO:1099, SEQ ID NO:1181, SEQ ID NO:1291, SEQ ID NO:1410, SEQ ID NO:1530, SEQ ID NO:1641, SEQ ID NO:1750 and SEQ ID NO:1929, or a functional variant thereof.

According to a further embodiment, the present invention discloses the method as defined in any of the above, wherein the Legume SELF PRUNING (SP) gene encodes a polypeptide sequence selected from the group consisting of: at least 75% identity to any one of Peanut polypeptide SEQ ID NO:1026, SEQ ID NO:1099, SEQ ID NO:1181, SEQ ID NO:1291, SEQ ID NO:1410, SEQ ID NO:1530, SEQ ID NO:1641, SEQ ID NO:1750 and SEQ ID NO:1929, or a functional variant thereof.

According to a further embodiment, the present invention discloses the method as defined in any of the above, wherein said modified plant is semi-determinant.

According to a further embodiment, the present invention discloses the method as defined in any of the above, wherein said modified plant has determinant growth habit.

According to a further embodiment, the present invention discloses the method as defined in any of the above, wherein said modified plant flowers earlier than a corresponding control Legume plant lacking said mutated SP gene.

According to a further embodiment, the present invention discloses the method as defined in any of the above, wherein said modified plant exhibits improved earliness as compared to a corresponding control Legume plant lacking said mutated SP gene.

According to a further embodiment, the present invention discloses the method as defined in any of the above, wherein said modified plant exhibits suppressed sympodial shoot termination as compared to a corresponding control Legume plant lacking said mutated SP gene.

According to a further embodiment, the present invention discloses the method as defined in any of the above, wherein said modified plant exhibits similar sympodial shoot termination as compared to a corresponding control Legume plant lacking said mutated SP gene.

According to a further embodiment, the present invention discloses the method as defined in any of the above, wherein said modified plant exhibits suppressed or reduced day-length sensitivity as compared to a corresponding control Legume plant lacking said mutated SP gene.

According to a further embodiment, the present invention discloses a modified Legume plant, plant part or plant cell produced by the method as defined in any of the above, wherein said modified plant does not comprise a transgene.

According to a further embodiment, the present invention discloses a plant part, plant cell, plant pod or plant seed of a modified Legume plant produced by the method as defined in any of the above.

According to a further embodiment, the present invention discloses a tissue culture of regenerable cells, protoplasts or callus obtained from the modified Legume plant produced by the method as defined in any of the above.

According to a further embodiment, the present invention discloses the method as defined in any of the above, wherein said at least one domestication trait is selected from the group consisting of reduced flowering time, earliness, synchronous flowering, reduced day-length sensitivity, determinant or semi-determinant architecture, early termination of sympodial cycling, earlier axillary shoot flowering, compact growth habit, reduced height, reduced number of sympodial units, adaptation to mechanical harvest, higher harvest index and any combination thereof.

According to a further embodiment, the present invention discloses an isolated polynucleotide sequence comprising at least 75% identity to a Legume SELF PRUNING (SP) sequence selected from the group consisting of SEQ ID NO:1, SEQ ID NO:113, SEQ ID NO:239, SEQ ID NO:340, SEQ ID NO:464, SEQ ID NO:616, SEQ ID NO:796, SEQ ID NO:1025, SEQ ID NO:1098, SEQ ID NO:1180, SEQ ID NO:1290, SEQ ID NO:1409, SEQ ID NO:1529, SEQ ID NO:1640, SEQ ID NO:1749 and SEQ ID NO:1928.

According to a further embodiment, the present invention discloses an isolated polypeptide sequence comprising at least 75% identity to a Legume SELF PRUNING (SP) sequence selected from the group consisting of SEQ ID NO:2, SEQ ID NO:114, SEQ ID NO:240, SEQ ID NO:341, SEQ ID NO:465, SEQ ID NO:617, SEQ ID NO:797, SEQ ID NO:1026, SEQ ID NO:1099, SEQ ID NO:1181, SEQ ID NO:1291, SEQ ID NO:1410, SEQ ID NO:1530, SEQ ID NO:1641, SEQ ID NO:1750 and SEQ ID NO:1929.

According to a further embodiment, the present invention discloses an isolated nucleotide sequence comprising at least 75% sequence identity to a Legume SELF PRUNING (SP)-targeted gRNA sequence selected from the group consisting of SEQ ID NO:3-112, SEQ ID NO:115-238, SEQ ID NO:241-339, SEQ ID NO:342-463, SEQ ID NO:466-615, SEQ ID NO:618-795, SEQ ID NO:798-1024, SEQ ID NO:1027-1097, SEQ ID NO:1100-1179, SEQ ID NO:1182-1289, SEQ ID NO:1292-1408, SEQ ID NO:1411-1528, SEQ ID NO:1531-1639, SEQ ID NO:1642-1748, SEQ ID NO:1751-1927 and SEQ ID NO:1930-2051.

According to a further embodiment, the present invention discloses harvestable parts of a modified Legume plant as defined in any of the above, wherein said harvestable parts are preferably shoot biomass and/or seeds.

According to a further embodiment, the present invention discloses products derived from a modified comprising at least 75% sequence identity to plant as defined in any of the above, and/or from harvestable parts of a modified Legume plant as defined in any of the above.

According to a further embodiment, the present invention discloses use of a nucleic acid encoding a polypeptide comprising at least 75% sequence identity to the sequence as defined in SEQ ID NO:2, SEQ ID NO:114, SEQ ID NO:240, SEQ ID NO:341, SEQ ID NO:465, SEQ ID NO:617, SEQ ID NO:797, SEQ ID NO:1026, SEQ ID NO:1099, SEQ ID NO:1181, SEQ ID NO:1291, SEQ ID NO:1410, SEQ ID NO:1530, SEQ ID NO:1641, SEQ ID NO:1750 and SEQ ID NO:1929, in enhancing yield and/or domestication, in Legume plants, relative to control plants.

In order to understand the invention and to see how it may be implemented in practice, a plurality of preferred embodiments will now be described, by way of non-limiting example only, with reference to the following examples.

### EXAMPLE 1

Production of Legume plants with improved domestication traits by targeted genome editing

Production of Legume lines with mutated sp gene may be achieved by at least one of the following breeding/cultivation schemes:

### Scheme 1

line stabilization by self pollination

Generation of F6 parental lines

Genome editing of parental lines

Crossing edited parental lines to generate an F1 hybrid plant

### Scheme 2

Identifying genes/alleles of interest

Designing gRNA

Transformation of plants with Cas9+gRNA constructs

Screening and identifying editing events

Genome editing of parental lines

It is noted that line stabilization may be performed by the following:

Induction of male flowering on plants

Self pollination

According to some embodiments of the present invention, line stabilization requires about 6 self-crossing 16 generations) and done through a single seed descent (SSD) approach.

F1 hybrid seed production: Novel hybrids are produced by crosses between different Legume strains.

According to a further aspect of the current invention, shortening line stabilization is performed by Doubled Haploids (DH). More specifically, the CRISPR-Cas9 system is transformed into microspores to achieve DH homozygous parental lines. A doubled haploid (DH) is a genotype formed when haploid cells undergo chromosome doubling. Artificial production of doubled haploids is important in plant breeding. It is herein acknowledged that conventional inbreeding procedures take about six generations to achieve approximately complete homozygosity, whereas doubled haploidy achieves it in one generation.

It is within the scope of the current invention that genetic markers specific for Legumes are developed and provided by the current invention:

Genotyping markers—germplasm used in the current invention is genotyped using molecular markers, in order to allow a more efficient breeding process and identification of the SP editing event.

It is further within the scope of the current invention that allele and genetic variation is analyzed for the Legume strains used.

Reference is now made to optional stages that have been used for the production of mutated SP Legume plants by genome editing:

Stage 1: Identifying Peanut SP genes.

SP orthologues have herein been identified in Peanut. These homologous genes have been sequenced and mapped. Peanut SP genes as set forth in SEQ ID NO:1025, SEQ ID NO:1098, SEQ ID NO:1180, SEQ ID NO:1290, SEQ ID NO:1409, SEQ ID NO:1529, SEQ ID NO:1640, SEQ ID NO:1749 and SEQ ID NO:1928.

Stage 2: Designing and synthesizing gRNA molecules corresponding to the sequence targeted for editing, i.e. sequences of each of the Peanut SP genes. It is noted that the editing event is preferably targeted to a unique restriction site sequence to allow easier screening for plants carrying an editing event within their genome. According to some aspects of the invention, the nucleotide sequence of the gRNAs should be completely compatible with the genomic sequence of the target gene. Therefore, for example, suitable gRNA molecules should be constructed for different SP homologues of different Legume strains.

Reference is now made to sequences of gRNA molecules targeted for silencing Peanut SP genes. Specifically, gRNA sequences SEQ ID NO:1027-1097, SEQ ID NO:1100-1179, SEQ ID NO:1182-1289, SEQ ID NO:1292-1408, SEQ ID NO:1411-1528, SEQ ID NO:1531-1639, SEQ ID NO:1642-1748, SEQ ID NO:1751-1927 and SEQ ID NO:1930-2051 are targeted for Peanut SP genes AhSP1-AhSP9, respectively, comprising sequence as set forth in SEQ ID NO:1025, SEQ ID NO:1098, SEQ ID NO:1180, SEQ ID NO:1290, SEQ ID NO:1409, SEQ ID NO:1529, SEQ ID NO:1640, SEQ ID NO:1749 and SEQ ID NO:1928, respectively. The term ‘PAM’ refers hereinafter to Protospacer Adjacent Motif, which is a 2-6 base pair DNA sequence immediately following the DNA sequence targeted by the Cas9 nuclease in the CRISPR bacterial adaptive immune system.

Reference is made to Table 1 presenting a summary of sequences within the scope of the current invention.

TABLE 2
Summary of Peanut (Arachis hypogaea) sequences within the scope of the present invention

| | AhSP1 | AhSP2 | AhSP3 | AhSP4 | AhSP5 | AhSP6 | AhSP7 | Ah SP8 | AhSP9 |
|---|---|---|---|---|---|---|---|---|---|
| Sequence | SEQ ID | SEQ ID | SEQ ID | SEQ ID | SEQ ID | SEQ ID | SEQ ID | SEQ ID | SEQ ID |
| type | NO | NO | NO | NO | NO | NO | NO | NO | NO |
| Genomic | 1025 | 1098 | 1180 | 1290 | 1409 | 1529 | 1640 | 1749 | 1928 |
| sequence | | | | | | | | | |
| Amino acid | 1026 | 1097 | 1181 | 1291 | 1410 | 1530 | 1641 | 1750 | 1929 |
| sequence | | | | | | | | | |
| gRNA | 1027- | 1100- | 1182- | 1292- | 1411- | 1531- | 1642- | 1751- | 1930- |
| sequence | 1097 | 1179 | 1289 | 1408 | 1528 | 1639 | 1748 | 1927 | 2051 |

The above gRNA molecules have been cloned into suitable vectors and their sequence has been verified. In addition different Cas9 versions have been analyzed for optimal compatibility between the Cas9 protein activity and the gRNA molecule in the Legume plant.

The efficiency of the designed gRNA molecules have been validated by transiently transforming Legume tissue culture. A plasmid carrying a gRNA sequence together with the Cas9 gene has been transformed into Legumes protoplasts. The protoplast cells have been grown for a short period of time and then were analyzed for existence of genome editing events. The positive constructs have been subjected to the herein established stable transformation protocol into Legume plant tissue for producing genome edited Legume plants in SP genes.

Stage 3: Transforming Legume plants using Agrobacterium or biolistics (gene gun) methods. For Agrobacterium and bioloistics, a DNA plasmid carrying (Cas9+gene specific gRNA) can be used. A vector containing a selection marker, Cas9 gene and relevant gene specific gRNA's is constructed. For biolistics, Ribonucleoprotein (RNP) complexes carrying (Cas9 protein+gene specific gRNA) are used. RNP complexes are created by mixing the Cas9 protein with relevant gene specific gRNA's.

According to some embodiments of the present invention, transformation of various Legume tissues was performed using particle bombardment of:
DNA vectors
Ribonucleoprotein complex (RNP's)

According to further embodiments of the present invention, transformation of various Legume tissues was performed using Agrobacterium (Agrobacterium tumefaciens) by:

Regeneration-based transformation

Floral-dip transformation

Seedling transformation

Transformation efficiency by A. tumefaciens has been compared to the bombardment method by transient GUS transformation experiment. After transformation, GUS staining of the transformants has been performed.

Screening for CRISPR/Cas9 gene editing events has been performed by at least one of the following analysis methods:

Restriction Fragment Length Polymorphism (RFLP)

Next Generation Sequencing (NGS)

PCR fragment analysis

Fluorescent-tag based screening

High resolution melting curve analysis (HRMA)

### REFERENCES

Tingdong Li, Xinping Yang, Yuan Yu, Xiaomin Si, Xiawan Zhai, Huawei Zhang, Wenxia Dong, Caixia Gao & Cao Xu. “Domestication of wild tomato is accelerated by genome editing” Nature Biotechnology 36 (2018): 1160-1163.
Agustin Zsögön, Tomáš Čermák, Emmanuel Rezende Naves, Marcela Morato Notini, Kai H Edel, Stefan Weinl, Luciano Freschi, Daniel F Voytas, Jörg Kudla & Lázaro Eustáquio Pereira Peres. “De novo domestication of wild tomato using genome editing”. Nature Biotechnology 36 (2018): 1211-1216.
Zachary H. Lemmon, Nathan T. Reem, Justin Dalrymple, Sebastian Soyk, Kerry E. Swartwood, Daniel Rodriguez-Leal, Joyce Van Eck & Zachary B. Lippman. “Rapid improvement of domestication traits in an orphan crop by genome editing”. Nature Plants 4 (2018): 766-770.
Xie Kabin, and Yinong Yang. “RNA-guided genome editing in plants using a CRISPR-Cas system” Molecular plant 6.6 (2013): 1975-1983.

1. A method for producing a modified Peanut plant exhibiting at least one improved domestication trait, wherein said method comprises steps of introducing by targeted genome editing a loss of function mutation in a Peanut (Arachis hypogaea) SELF PRUNING (SP) gene, said improved domestication trait is relative to a corresponding Peanut plant lacking the loss of function mutation.

2. The method according to claim 1, wherein said Peanut (Arachis hypogaea) SP gene is selected from AhSP1-AhSP9 comprising a nucleic acid sequence with at least 75% sequence identity to a sequence selected from SEQ ID NO:1025, SEQ ID NO:1098, SEQ ID NO:1180, SEQ ID NO:1290, SEQ ID NO:1409, SEQ ID NO:1529, SEQ ID NO:1640, SEQ ID NO:1749 and SEQ ID NO:1928, or a functional variant thereof and any combination thereof.

3. The method according to claim 1, wherein said method comprises steps of:
a. identifying the SP gene in a Peanut (Arachis hypogaea) plant;
b. synthetizing at least one guide RNA (gRNA) comprising a nucleotide sequence complementary to said at least one identified Peanut SP gene;
c. transforming the Peanut plant cells with a construct comprising (a) Cas nucleotide sequence operably linked to said at least one gRNA, or (b) a ribonucleoprotein (RNP) complex comprising Cas protein and said at least one gRNA;
d. screening the genome of said transformed predetermined Peanut plant cells for induced targeted loss of function mutation in said at least one Peanut SP gene;
e. regenerating Peanut plants carrying said loss of function mutation in at least one of said Peanut SP gene; and
f. screening said regenerated plants for a Peanut plant with improved domestication trait.

4. The method according to claim 3, wherein said step of screening the genome of said transformed plant cells for induced targeted loss of function mutation further comprises steps of obtaining a nucleic acid sample of said transformed plant and performing a nucleic acid amplification and optionally restriction enzyme digestion to detect a mutation in said at least one of said Peanut SP gene.

5. The method according to claim 1, wherein said SP gene is selected from the group consisting of Peanut SP gene comprising a sequence having at least 75% identity to a sequence selected from SEQ ID NO:1025, SEQ ID NO:1098, SEQ ID NO:1180, SEQ ID NO:1290, SEQ ID NO:1409, SEQ ID NO:1529, SEQ ID NO:1640, SEQ ID NO:1749 and SEQ ID NO:1928, or a functional variant thereof and any combination thereof.

6. The method according to claim 1, wherein said mutation is introduced using CRISPR (Clustered Regularly Interspaced Short Palindromic Repeats) and CRISPR-associated (Cas) gene (CRISPR/Cas), Transcription activator-like effector nuclease (TALEN), Zinc Finger Nuclease (ZFN), meganuclease or any combination thereof.

7. The method according to claim 6, wherein said Cas gene is selected from the group consisting of Cas9, Cas12, Cas13, Cas14, CasX, CasY, Csn1, Cpf1 and any combination thereof.

8. The method according to claim 1, wherein the mutated SP gene is a CRISPR/Cas9-induced heritable mutated allele.

9. The method according to claim 1, wherein said mutation is a missense mutation, nonsense mutation, insertion, deletion, indel, substitution or duplication.

10. The method according to claim 1, wherein said mutation is a silencing mutation, a knockdown mutation, a knockout mutation, a loss of function mutation or any combination thereof.

11. The method according to claim 1, wherein said mutation is generated in planta via introduction of a construct comprising (a) Cas DNA and gRNA sequence selected from the group consisting of SEQ ID NO:1027-1097, SEQ ID NO:1100-1179, SEQ ID NO:1182-1289, SEQ ID NO:1292-1408, SEQ ID NO:1411-1528, SEQ ID NO:1531-1639, SEQ ID NO:1642-1748, SEQ ID NO:1751-1927 and SEQ ID NO:1930-2051, for said at least one Peanut SP gene, and any combination thereof, or (b) a ribonucleoprotein (RNP) complex comprising Cas protein and gRNA sequence selected from the group consisting of SEQ ID NO:1027-1097, SEQ ID NO:1100-1179, SEQ ID NO:1182-1289, SEQ ID NO:1292-1408, SEQ ID NO:1411-1528, SEQ ID NO:1531-1639, SEQ ID NO:1642-1748, SEQ ID NO:1751-1927 and SEQ ID NO:1930-2051, for said at least one Peanut SP gene, and any combination thereof.

12. The method according to claim 1, wherein said mutation in said Peanut SP genes is generated in planta via introduction of a construct comprising (a) Cas DNA and gRNA sequence selected from the group consisting of SEQ ID NO:1027-1097, SEQ ID NO:1100-1179, SEQ ID NO:1182-1289, SEQ ID NO:1292-1408, SEQ ID NO:1411-1528, SEQ ID NO:1531-1639, SEQ ID NO:1642-1748, SEQ ID NO:1751-1927 and SEQ ID NO:1930-2051 for Peanut SEQ ID NO:1025, SEQ ID NO:1098, SEQ ID NO:1180, SEQ ID NO:1290, SEQ ID NO:1409, SEQ ID NO:1529, SEQ ID NO:1640, SEQ ID NO:1749 and SEQ ID NO:1928, respectively, or (b) a ribonucleoprotein (RNP) complex comprising Cas protein and gRNA sequence selected from the group consisting of SEQ ID NO:1027-1097, SEQ ID NO:1100-1179, SEQ ID NO:1182-1289, SEQ ID NO:1292-1408, SEQ ID NO:1411-1528, SEQ ID NO:1531-1639, SEQ ID NO:1642-1748, SEQ ID NO:1751-1927 and SEQ ID NO:1930-2051 for Peanut SEQ ID NO:1025, SEQ ID NO:1098, SEQ ID NO:1180, SEQ ID NO:1290, SEQ ID NO:1409, SEQ ID NO:1529, SEQ ID NO:1640, SEQ ID NO:1749 and SEQ ID NO:1928, respectively.

13. The method according to claim 12, wherein said gRNA sequence comprises a 3′ NGG Protospacer Adjacent Motif (PAM).

14. The method according to claim 12, wherein said construct is introduced into the plant cells via Agrobacterium infiltration, virus based plasmids for delivery of the genome editing molecules or mechanical insertion such as polyethylene glycol (PEG) mediated DNA transformation, electroporation or gene gun biolistics.

15. The method according to claim 13, wherein said gRNA sequence comprises a 3′ NGG Protospacer Adjacent Motif (PAM).

16. The method according to claim 13, wherein said construct is introduced into the plant cells via Agrobacterium infiltration, virus based plasmids for delivery of the genome editing molecules or mechanical insertion such as polyethylene glycol (PEG) mediated DNA transformation, electroporation or gene gun biolistics.
